(19) United States
(12) Patent Application Publication
(10) Pub. No.: US 2024/0309469 A1
(43) Pub. Date: Sep. 19, 2024

(54) METHODS TO DETECT AND TREAT A FUNGAL INFECTION

(71) Applicants: DUKE UNIVERSITY, Durham, NC (US); THE U.S. GOVERNMENT AS REPRESENTED BY THE DEPT. OF VETERAN AFFAIRS, Washington, DC (US)

(72) Inventors: Julie STEINBRINK, Durtham, NC (US); Micah MCCLAIN, Durham, NC (US); Rachel MYERS, Durham, NC (US); Melissa JOHNOSN, Durham, NC (US); Ephraim TSALIK, Cary, NC (US); Barbara ALEXANDER, Durham, NC (US); Christopher WOODS, Durham, NC (US)

(21) Appl. No.: 18/264,077

(22) PCT Filed: Feb. 4, 2022
(86) PCT No.: PCT/US2022/015195

§ 371 Date: Aug. 3, 2023

Related U.S. Application Data
(60) Provisional application No. 63/146,212, filed on Feb. 5, 2021.

(51) Int. Cl. C12Q 1/6895; A61K 31/135; A61K 31/138; A61K 31/4196; A61K 31/4439; A61K 31/496; A61K 31/498; A61K 31/506; A61K 31/513; A61K 31/7048; A61K 38/12; C12Q 1/6874; C12Q 1/689; C12Q 1/70

(52) U.S. Cl. CPC C12Q 1/6895; A61K 31/135; A61K 31/138; A61K 31/4196; A61K 31/4439; A61K 31/496; A61K 31/498; A61K 31/506; A61K 31/513; A61K 31/7048; A61K 38/12; C12Q 1/689; C12Q 1/701; C12Q 1/6874; C12Q 2600/106; C12Q 2600/158; C12Q 2600/178

(57) ABSTRACT

The present disclosure provides methods for determining whether a subject has a fungal infection such as candidemia, or is at risk of developing the same, and methods of treating the subject based on the determination. This determining may include rapid detection of one or multiple pathogen classes at once, such as fungal, viral and bacterial. Systems useful for the same are also provided.

### CROSS-REFERENCE TO RELATED APPLICATIONS

This application claims the benefit of U.S. Provisional Patent Application Ser. No. 63/146,212, filed Feb. 5, 2021, the disclosure of which is incorporated by reference herein in its entirety.

### FEDERAL FUNDING LEGEND

This invention was made with government support under Federal Grant no. R21AI132978-01 awarded by the National Institute of Allergy and Infectious Diseases (NIH/NIAID). The government has certain rights to this invention.

### BACKGROUND

Candidemia is one of the most common nosocomial bloodstream infections in the United States and causes significant morbidity and mortality in hospitalized patients. Improved rapid diagnostics capable of differentiating Candidemia from other causes of febrile illness in the hospitalized patient are of paramount importance. Pathogen class-specific biomarker-based diagnostics such as those focusing on host gene expression patterns in circulating leukocytes may offer a promising alternative.

US 2016/0194715 to Zaas et al. discusses methods of identifying fungal infection such as candidiasis by proteomic assay of a peripheral blood sample.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Provided herein according to some aspects is a method for classifying a subject, comprising: (a) obtaining a biological sample from the subject; (b) measuring on a platform a signature indicative of a fungal infection, and optionally one or more of a bacterial infection, a viral infection, healthy and/or non-infectious illness in the biological sample, said signature(s) comprising gene expression levels of a pre-defined set of genes; (c) entering the gene expression levels into a fungal classifier, and optionally one or more additional classifiers selected from a bacterial infection classifier, a viral classifier, and a control classifier (healthy and/or non-infectious illness), said classifier(s) comprising pre-defined weighting values (i.e., coefficients) for each of the genes of the pre-defined set of genes for the platform; and (d) classifying the subject as having a fungal infection, and/or a bacterial infection, a viral infection, or a control, based upon said gene expression levels and the classifier(s).

In some embodiments, the method comprises normalizing the gene expression levels to generate normalized gene expression values, and the entering comprises entering the normalized gene expression values into the classifier(s); and the classifying comprises calculating the probability for the fungal infection, and optionally a bacterial infection, a viral infection, or a control based upon said normalized gene expression values and the classifier(s).

In some embodiments, the method further comprises generating a report assigning the subject a score indicating the probability of the fungal infection, and optionally the bacterial infection, viral infection, healthy and/or non-infectious illness.

In some embodiments, the method further comprises: (e) administering an appropriate therapy to the subject based on the classifying.

In some embodiments, the pre-defined set of genes is a set of from 1, 5, 10, 15, or 20 to 30, 40, 50, 60 or 70 genes. In some embodiments, the pre-defined set of genes is a set of from 1, 5, 10, 15, or 20 to 30, 40, 50, 60 or 70 genes listed in Tables 1-5. In some embodiments, the pre-defined set of genes is a set of from 1, 5, or 10, to 15, 20, 25, 30 or 33 genes listed in Tables 6-10 (e.g., selected from the genes listed in bold type in Tables 6-10).

In some embodiments, the subject has symptoms of an infection (e.g., fever). In some embodiments, the subject has symptoms of sepsis.

In some embodiments, the biological sample is selected from the group consisting of peripheral blood, sputum, cerebrospinal fluid, urine, nasopharyngeal swab, nasopharyngeal wash, bronchoalveolar lavage, endotracheal aspirate, and combinations thereof. In some embodiments, the biological sample comprises a peripheral blood sample. In some embodiments, the biological sample comprises a bronchoalveolar lavage.

In some embodiments, the measuring comprises or is preceded by one or more steps of purifying cells from the sample, breaking the cells of the sample, and isolating RNA from the sample.

In some embodiments, the measuring comprises PCR amplification, isothermal amplification, sequencing and/or nucleic acid probe hybridization. In some embodiments, the platform comprises an array platform, a thermal cycler platform (e.g., multiplexed and/or real-time PCR platform), a hybridization and multi-signal coded (e.g., fluorescence) detector platform, a nucleic acid mass spectrometry platform, a nucleic acid sequencing platform, an isothermal amplification platform, or a combination thereof.

In some embodiments, the fungal infection comprises a yeast, such as Candida, Trichosporon, or Cryptococcus.

In some embodiments, the fungal classifier is/was produced by a process comprising: (i) obtaining a biological sample from a plurality of subjects known to be suffering from a fungal infection; (ii) obtaining a biological sample from a plurality of non-hospitalized healthy controls and/or a plurality of subjects known to be suffering from a non-infectious illness; (iii) measuring on the platform the gene expression levels of a plurality of genes in each of the samples from steps (i) and (ii); (iv) normalizing the gene expression levels obtained in step (iii) to generate normalized gene expression values; and (f) generating the fungal classifier.

In some embodiments, the fungal classifier is/was produced by a process comprising: (i) obtaining a biological sample from a plurality of subjects known to be suffering from a fungal infection; (ii) obtaining a biological sample from a plurality of subjects known to be suffering from a bacterial infection; (iii) measuring on the platform the gene expression levels of a plurality of genes in each of the samples from steps (i) and (ii); (iv) normalizing the gene expression levels obtained in step (iii) to generate normalized gene expression values; and (f) generating the fungal classifier.

In some embodiments, the fungal classifier is/was produced by a process comprising: (i) obtaining a biological sample from a plurality of subjects known to be suffering from a fungal infection; (ii) obtaining a biological sample from a plurality of subjects known to be suffering from a viral infection; (iii) measuring on the platform the gene expression levels of a plurality of genes in each of the samples from steps (i) and (ii); (iv) normalizing the gene expression levels obtained in step (iii) to generate normalized gene expression values; and (f) generating the fungal classifier.

In some embodiments, the generating comprises iteratively: (i) assigning a weight for each normalized gene expression value, entering the weight and expression value for each gene into a classifier (e.g., a linear regression classifier) equation and determining a score for outcome for each of the plurality of subjects, then (ii) determining the accuracy of classification for each outcome across the plurality of subjects, and then (iii) adjusting the weight until accuracy of classification is optimized, to provide said fungal classifier, bacterial classifier, viral classifier, and/or control classifier for the platform, wherein genes having a non-zero weight are included in the respective classifier, and optionally uploading components of each classifier (genes, weights and/or etiology threshold value) onto one or more databases.

Also provided according to some aspects is a method for detecting a fungal infection in a subject, comprising: providing a biological sample of the subject; and measuring on a platform differential expression of a pre-defined set of genes, said pre-defined set of genes comprising 5, 10, 15, 20, 25, or 30 to 50, 60, 70, 80, 90 or all 94 of the genes listed in Tables 1 to 5; such as 3, 5, 8, 10, 12, 15, 18, 20, 25, or all 29 of the genes listed in Table 1; and optionally 3, 5, 8, 10, 12, 15, or all 18 of the genes listed in Table 2; 3, 5, 8, 10, 12, 15, 18, or all 19 of the genes listed in Table 3; 3, 5, 8, 10, 12, 15, 18, or all 19 of the genes listed in Table 4; and/or 3, 4, 5, 6, 7, 8, 9, or all 10 of the genes listed in Table 5, or wherein said pre-defined set of genes comprises 5, 10, 15, 20, 25, 30, or all 33 of the genes (measurable, e.g., with oligonucleotide probes homologous to said genes) listed in Tables 6 to 10; such as 1, 2, 3, 4 or all 5 of the genes listed in Table 6; and optionally 1, 2, 3, 4, 5, 6, 7, 8 or all 9 of the genes listed in Table 7; 1, 2, 3, 4, 5, 6, 7 or all 8 of the genes listed in Table 8; 1, 2, 3, 4, 5, 6 or all 7 of the genes listed in Table 9; and/or 1, 2, 3 or all 4 of the genes listed in Table 10, or wherein said pre-defined set of genes comprises ITGA2B, MK167, and AZU1; and optionally HDAC4, DCAF15, SDHC, SAP30L, DNASE1, and DCAF15; PIGT, HERC6, and LY6E; SLC35EL1, WIPI2, RELL1, MAP1LC3B, CASZ1 and GABBR1; and/or RPS24 and CTSB, wherein the differential expression of the pre-defined set of genes indicates the presence or absence of the fungal infection in the subject.

In some embodiments, measuring comprises or is preceded by one or more steps of: purifying cells from said sample, breaking the cells of said sample, and isolating RNA from said sample.

In some embodiments, measuring comprises semi-quantitative PCR, isothermal amplification, and/or nucleic acid probe hybridization. In some embodiments, the platform comprises an array platform, a thermal cycler platform (e.g., multiplexed and/or real-time PCR platform), an isothermal amplification platform, a hybridization and multi-signal coded (e.g., fluorescence) detector platform, a nucleic acid mass spectrometry platform, a nucleic acid sequencing platform, or a combination thereof.

In some embodiments, the subject is suffering from symptoms of an infection (e.g., fever). In some embodiments, the subject is suffering from symptoms of sepsis.

In some embodiments, the method further comprises treating said subject for the fungal infection when the presence of the fungal infection is detected.

Further provided according to some aspects is a method of treating a fungal infection in a subject comprising administering to said subject an appropriate treatment regimen when said subject is determined to have a fungal infection by a method as taught herein. Also provided is the use of an appropriate treatment regimen for treating a fungal infection in a subject, when said subject is determined to have a fungal infection by a method as taught herein.

In some embodiments, the appropriate treatment regimen comprises administering an antifungal antibiotic. In some embodiments, the appropriate treatment regimen comprises administering a therapeutic agent selected from the group consisting of: echinocandins (e.g., caspofungin, micafungin, anidulafungin), azole antifungals (e.g., fluconazole, voriconazole, isavuconazole, posaconazole), polyenes (e.g., amphotericin B), pyrimidine analogues (e.g., 5-fluorocytosine (5-FC, or flucytosine)), APX001 (fosmanogepix), APX879, benzothioureas, clofazimine, hydrazycines (e.g., BHBM and B0), ibomycin, monoclonal antibody 18B7, resorcylate aminopyrazoles (e.g., Compound 112), sertraline, tamoxifen, VT-1598, and the like, including combinations thereof.

In some embodiments, the method further comprises monitoring the subject for efficacy of the appropriate treatment regimen by use of a method of detecting a fungal infection as taught herein.

Further provided according to some aspects is a system for detecting a fungal infection in a subject, comprising: at least one processor; a sample input circuit configured to receive a biological sample from the subject; a sample analysis circuit coupled to the at least one processor and configured to determine gene expression levels of the biological sample of a set of pre-determined genes indicative of the fungal infection; an input/output circuit coupled to the at least one processor; a storage circuit coupled to the at least one processor and configured to store data, parameters, and/or gene set(s); and a memory coupled to the processor and comprising computer readable program code embodied in the memory that when executed by the at least one processor causes the at least one processor to perform operations comprising: controlling/performing measurement via the sample analysis circuit of gene expression levels of the pre-defined set of genes in said biological sample; normalizing the gene expression levels to generate normalized gene expression values; retrieving from the storage circuit pre-defined weighting values (i.e., coefficients) for each of the genes of the pre-defined set of genes; calculating a likelihood of the fungal infection based upon weighted values of the normalized gene expression values; and controlling output via the input/output circuit of a determination of the presence or absence of the fungal infection.

In some embodiments, the pre-defined set of genes comprises 5, 10, 15, 20, 25, or 30 to 50, 60, 70, 80, 90 or all 94 of the genes listed in Tables 1 to 5; such as 3, 5, 8, 10, 12, 15, 18, 20, 25, or all 29 of the genes listed in Table 1; and optionally 3, 5, 8, 10, 12, 15, or all 18 of the genes listed in Table 2; 3, 5, 8, 10, 12, 15, 18, or all 19 of the genes listed in Table 3; 3, 5, 8, 10, 12, 15, 18, or all 19 of the genes listed in Table 4; and/or 3, 4, 5, 6, 7, 8, 9, or all 10 of the genes listed in Table 5, or wherein said pre-defined set of genes comprises 5, 10, 15, 20, 25, 30, or all 33 of the genes listed in Tables 6 to 10; such as 1, 2, 3, 4 or all 5 of the genes listed in Table 6; and optionally 1, 2, 3, 4, 5, 6, 7, 8 or all 9 of the genes listed in Table 7; 1, 2, 3, 4, 5, 6, 7 or all 8 of the genes listed in Table 8; 1, 2, 3, 4, 5, 6 or all 7 of the genes listed in Table 9; and/or 1, 2, 3 or all 4 of the genes listed in Table 10, or wherein said pre-defined set of genes comprises ITGA2B, MK167, and AZU1; and optionally HDAC4, DCAF15, SDHC, SAP30L, DNASE1, and DCAF15; PIGT, HERC6, and LY6E; SLC35ET, WIPI2, RELL1, MAP1LC3B, CASZ1 and GABBR1; and/or RPS24 and CTSB.

In some embodiments, the system comprises computer readable code to transform quantitative, or semi-quantitative, detection of gene expression to a cumulative score or probability of the fungal infection.

In some embodiments, the system comprises an array platform, a thermal cycler platform (e.g., multiplexed and/or real-time PCR platform), a hybridization and multi-signal coded (e.g., fluorescence) detector platform, a nucleic acid mass spectrometry platform, a nucleic acid sequencing platform, an isothermal amplification platform, or a combination thereof.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIG. 1 is a schematic showing the experimental design for the breakdown of discovery and validation cohorts by infection phenotype in accordance with one embodiment of the present disclosure.

FIG. 2A shows differentially expressed genes (adj P<0.05) in response to different infectious phenotypes. All genes, infection phenotypes compared to all others.

FIG. 2B shows differentially expressed genes (adj P<0.05) in response to different infection phenotypes. All genes, Candida compared to each other phenotype.

FIG. 3 presents graphs showing multinomial gene expression classifiers in accordance with embodiments of the present disclosure. Panel A. ROCs of the multinomial classifier performance for each infection phenotype in the discovery cohort. Panel B. Boxplots demonstrating predictive probability of the classifier for each infection phenotype in the discovery cohort. Panel C. ROCs of the multinomial classifier performance for each infection phenotype in the validation cohort. Panel D. Boxplots demonstrating predictive probability of the classifier for each infection phenotype in the validation cohort.

FIG. 4 presents graphs showing validation cohorts in accordance with embodiments of the present disclosure. ROCs (Panel A) and Boxplots (Panel B) of the multinomial classifier performance for each infection phenotype in the Tsalik, et al. cohort. ROCs (Panel C) and Boxplots (Panel D) of the multinomial classifier performance for each infection phenotype in the Ramilo, et al. cohort. ROCs (Panel E) and Boxplots (Panel F) of the multinomial classifier performance for each infection phenotype in the in vitro cohort. Infection class as established by the classifier was determined by the phenotype with the highest predictive probability per subject.

FIG. 5 is a block diagram of a classification system and/or computer program product that may be used in a platform in accordance with the present invention. A classification system and/or computer program product 1100 may include a processor subsystem 1140, including one or more Central Processing Units (CPU) on which one or more operating systems and/or one or more applications run. While one processor 1140 is shown, it will be understood that multiple processors 1140 may be present, which may be either electrically interconnected or separate. Processor(s) 1140 are configured to execute computer program code from memory devices, such as memory 1150, to perform at least some of the operations and methods described herein. The storage circuit 1170 may store databases which provide access to the data/parameters/classifiers used by the classification system 1110 such as the signatures, weights, thresholds, etc. An input/output circuit 1160 may include displays and/or user input devices, such as keyboards, touch screens and/or pointing devices. Devices attached to the input/output circuit 1160 may be used to provide information to the processor 1140 by a user of the classification system 1100. Devices attached to the input/output circuit 1160 may include networking or communication controllers, input devices (keyboard, a mouse, touch screen, etc.) and output devices (printer or display). An optional update circuit 1180 may be included as an interface for providing updates to the classification system 1100 such as updates to the code executed by the processor 1140 that are stored in the memory 1150 and/or the storage circuit 1170. Updates provided via the update circuit 1180 may also include updates to portions of the storage circuit 1170 related to a database and/or other data storage format which maintains information for the classification system 1100, such as the signatures, weights, thresholds, etc. The sample input circuit 1110 provides an interface for the classification system 1100 to receive biological samples to be analyzed. The sample processing circuit 1120 may further process the biological sample within the classification system 1100 so as to prepare the biological sample for automated analysis.

### DETAILED DESCRIPTION

The disclosures of all patent references cited herein are hereby incorporated by reference to the extent they are consistent with the disclosure set forth herein.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a,” “an” and “the” are used herein to refer to one or to more than one (i.e., at least one) of the grammatical object of the article. By way of example, “an element” means at least one element and can include more than one element.

“About” is used to provide flexibility to a numerical range endpoint by providing that a given value may be slightly above or slightly below (e.g., by 2%, 5%, 10% or 15%) the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “signature” as used herein refers to a set of biological analytes and the measurable quantities of said analytes whose particular combination signifies the presence or absence of the specified biological state. These signatures are discovered in a plurality of subjects with known status (e.g., with a confirmed bacterial infection, viral infection, fungal infection, or control (healthy and/or non-infectious illness)), and are discriminative (individually or jointly) of one or more categories or outcomes of interest. These measurable analytes, also known as biological markers, can be (but are not limited to) gene expression levels, protein or peptide levels, or metabolite levels. See also US 2015/0227681 to Courchesne et al.; US 2016/0153993 to Eden et al.

In some embodiments as disclosed herein, the “signature” is a particular combination of genes whose expression levels, when incorporated into a classifier as taught herein, discriminate a condition such as a fungal infection. See, for example, the Examples provided hereinbelow. However, the signature may be processed/interpreted in other manners, such as those noted in US 2015/0227681 to Courchesne et al. and US 2016/0153993 to Eden et al. As a non-limiting example, U.S. Pat. No. 10,533,224 to Khatri et al. discusses comparison of biomarker levels to reference value ranges of a non-infected control subject, such as time-matched reference value ranges, and the use of a geometric mean of the biomarker expression levels compared to control reference values for the biomarkers, to discriminate a condition or biological state.

As used herein, the terms “classifier” and “predictor” are used interchangeably and refer to a mathematical function that uses the values of the signature (e.g., gene expression levels for a defined set of genes) and a pre-determined coefficient (or weight) for each signature component to generate scores for a given observation or individual patient for the purpose of assignment to a category. The classifier may be linear and/or probabilistic. A classifier is linear if scores are a function of summed signature values weighted by a set of coefficients. Furthermore, a classifier is probabilistic if the function of signature values generates a probability, a value between 0 and 1.0 (or between 0 and 100%) quantifying the likelihood that a subject or observation belongs to a particular category or will have a particular outcome, respectively. Probit regression and logistic regression are examples of probabilistic linear classifiers that use probit and logistic link functions, respectively, to generate a probability.

A classifier as taught herein may be obtained by a procedure known as “training,” which makes use of a set of data containing observations with known category membership (e.g., fungal, viral, bacterial, control, etc.). Specifically, training seeks to find the optimal coefficient (i.e., weight) for each component of a given signature (e.g., gene expression level components), as well as an optimal signature, where the optimal result is determined by the highest achievable classification accuracy.

“Classifying” or “classification” as used herein refers to a method of assigning a subject suffering from or at risk for a biological state such an infection (e.g., a fungal infection) to one or more categories or outcomes (e.g., a patient is infected with a pathogen or is not infected). The outcome, or category, is determined by the value of the scores provided by the classifier, which may be compared to a cut-off or threshold value, confidence level, or limit. In other scenarios, the probability of belonging to a particular category may be given (e.g., if the classifier reports probabilities).

As used herein, the term “indicative” when used with gene expression levels, means that the gene expression levels are up-regulated or down-regulated, altered, or changed compared to the expression levels in alternative biological states or control. The term “indicative” when used with protein levels means that the protein levels are higher or lower, increased or decreased, altered, or changed compared to the standard protein levels or levels in alternative biological states.

In some embodiments, the classifier/classification is “agnostic” in that it is indicative of a general biological state, such as a fungal infection, a bacterial infection, a viral infection, or SIRS, but it does not provide an indication of a particular organism (genus and optionally species) as a cause of the state (e.g., a particular fungus or bacteria causing the infection).

As used herein, the terms “biomarker” or “biological markers” are used interchangeably and refer to a naturally occurring biological molecule present in a subject at varying concentrations useful in predicting the risk or incidence of a disease or a condition, such as a fungal infection. For example, the biomarker can be a protein or gene expression present in higher or lower amounts in a subject at risk for, or suffering from, a fungal infection such as candidemia. The biomarker can include, but is not limited to, nucleic acids, ribonucleic acids, or a polypeptide used as an indicator or marker for a biological state in the subject. In some embodiments, the biomarker comprises RNA. In other embodiments, the biomarker comprises DNA. In yet other embodiments, the biomarker comprises a protein. A biomarker may also comprise any naturally or non-naturally occurring polymorphism (e.g., single-nucleotide polymorphism (SNP)) or gene variant present in a subject that is useful in predicting the risk or incidence of a fungal infection such as candidemia.

As used herein, “treating,” “treatment,” “therapy” and/or “therapy regimen” refer to the clinical intervention made in response to a disease, disorder, physiological condition or biological state (e.g., fungal infection) manifested by a patient or to which a patient may be susceptible. The aim of treatment includes the alleviation or prevention/reduction of symptoms, slowing or stopping the progression or worsening of a disease, disorder, or condition and/or the remission of the disease, disorder or condition such as infection. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition (e.g., fungal infection such as candidemia). The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance (e.g., drug, therapy, etc.) to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “appropriate treatment regimen” or “appropriate therapy” refers to the standard of care needed to treat a specific disease or disorder. Often such regimens require the act of administering to a subject a therapeutic agent capable of producing a curative effect in a disease state. For example, therapeutic agents for treating a subject having a fungal infection (e.g., candidemia, a Cryptococcus infection, etc.) may include, for example, an antifungal antibiotic. Particular therapeutic agents for treating a subject having a fungal infection may include, but are not limited to, drugs such as echinocandins (e.g., caspofungin, micafungin, anidulafungin), azole antifungals (e.g., fluconazole, voriconazole, isavuconazole, posaconazole), polyenes (e.g., amphotericin B), pyrimidine analogues (e.g., 5-fluorocytosine (5-FC, or flucytosine)), APX001 (fosmanogepix), APX879, benzothioureas, clofazimine, hydrazycines (e.g., BHBM and B0), ibomycin, monoclonal antibody 18B7, resorcylate aminopyrazoles (e.g., Compound 112), sertraline, tamoxifen, VT-1598, and the like, including combinations thereof. See, e.g., Iyer et al., “Treatment strategies for cryptococcal infection: challenges, advances and future outlook,” Nature Reviews Microbiology 19, 454-466 (2021).

Treatment of a bacterial infection may comprise an antibiotic, which include, but are not limited to, penicillins, cephalosporins, fluroquinolones, tetracyclines, macrolides, and aminoglycosides. A therapeutic agent for treating a subject having a viral infection includes, but is not limited to, oseltamivir, RNAi antivirals, inhaled ribavirin, monoclonal antibody respigam, zanamivir, and neuraminidase blocking agents. The present disclosure contemplates the use of the methods taught herein to determine treatments with antifungals, antivirals or antibiotics that are not yet available.

Such regimens may also include administering to a subject a therapeutic agent capable of producing a reduction of symptoms associated with a disease or biological state. Examples of such therapeutic agents include, but are not limited to, NSAIDS, acetaminophen, anti-histamines, beta-agonists, anti-tussives or other medicaments that reduce the symptoms associated with the disease or infectious process.

The term “biological sample” as used herein includes, but is not limited to, a sample containing tissues, cells, and/or biological fluids isolated from a subject. Examples of biological samples include, but are not limited to, tissues, cells, biopsies, blood (e.g., peripheral blood), lymph, serum, plasma, cerebrospinal fluid, urine, saliva, mucus, tears, sputum, nasopharyngeal swab, nasopharyngeal wash, bronchoalveolar lavage, endotracheal aspirate, and the like. In some embodiments, the biological sample comprises peripheral blood. In some embodiments, the biological sample comprises bronchoalveolar lavage. A biological sample may be obtained directly from a subject (e.g., by blood or tissue sampling) or from a third party (e.g., received from an intermediary, such as a healthcare provider or lab technician).

The term “genetic material” refers to a material corresponding to that used to store genetic information in the nuclei or mitochondria of an organism's cells. Examples of genetic material include, but are not limited to, double-stranded and single-stranded DNA, cDNA, RNA, and mRNA.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient). In some embodiments, the subject comprises a human who is suffering from, or at risk of suffering from, a fungal infection such as candidemia. In some embodiments, the subject has symptoms of an infection (e.g., fever). In some embodiments, the subject has symptoms of sepsis.

“Sepsis” as used herein refers to organ dysfunction caused by a dysregulated host response to infection. See Singer, M. et al. The Third International Consensus Definitions for Sepsis and Septic Shock (Sepsis-3). JAMA 315, 801 (2016). Organ dysfunction may be determined, e.g., by an increase in the sequential organ failure assessment (also known as sepsis-related organ failure assessment, or SOFA) score of two or more points over baseline.

Unless otherwise defined, all technical terms used herein have the same meaning as commonly understood by one of ordinary skill in the art to which this disclosure belongs.

One aspect of the present disclosure provides a method for generating pathogen class-specific classifiers for a platform capable of identifying and differentiating fungal, viral, and/or bacterial infection across a variety of hosts with a high degree of accuracy, the method comprising, consisting of, or consisting essentially of (i) obtaining a biological sample from a plurality of subjects known to suffering from a fungal infection; (ii) obtaining a biological sample from a plurality of non-hospitalized healthy controls; (iii) measuring on the platform the gene expression levels of a plurality of genes in each of the samples from steps (i) and (ii); (iv) optionally normalizing the gene expression levels obtained in step (iii) to generate normalized gene expression values; and (f) generating one or more classifiers capable of identifying and differentiating a fungal infection across a variety of hosts with a high degree of accuracy.

In some embodiments, the method provides further obtaining biological samples from plurality of subjects suffering from viral and/or bacterial infections and/or non-infection illness (SIRS) for use in the generating step.

In some embodiments, the measuring comprises or is preceded by one or more steps of purifying cells from the sample, breaking the cells of the sample, and isolating RNA from the sample.

In some embodiments, the measuring comprises PCR, reverse transcription (of mRNA to cDNA), isothermal amplification, and/or nucleic acid probe hybridization.

A “fungal infection” as used herein refers to an infection (e.g., a blood infection, lung infection, etc.) of a host subject with a pathogenic fungus (e.g., yeast, mold, dematiaceous fungus). The fungus may include, but is not limited to, a fungus of the genus Candida (which causes candidemia and candidiasis), of the genus Cryptococcus (e.g., Cryptococcus neoformans), of the genus Aspergillus, of the genus Histoplasma (e.g., Histoplasma capsulatum), of the genus Pneumocystis, of the genus Coccidioides (e.g., Coccidioides immitis), of the genus Paracoccidioides (e.g., Paracoccidioides brasiliensis), of the genus Sporothrix (e.g., Sporothrix schenckii), etc.

In some embodiments, the fungus is a yeast, such as Candida, Trichosporon, or Cryptococcus. Representative species of Candida include, but are not limited to, Candida albicans, Candida glabrata, Candida tropicalis, Candida dubliniensis, Candida krusei, Candida lusitanae, Candida parapsilosis, and Candida zeylanoides. Representative species of Trichosporon include, but are not limited to, Trichosporon fungemia. Representative species of Cryptococcus include, but are not limited to, Cryptococcus neoformans and Cryptococcus gattii.

As used herein, the term “platform” or “technology” refers to an apparatus (e.g., instrument and associated parts, computer, computer-readable media comprising one or more databases as taught herein, reagents, etc.) that may be used to measure a signature, e.g., gene expression levels, in accordance with the present disclosure. Examples of platforms include, but are not limited to, an array platform, a thermal cycler platform (e.g., multiplexed and/or real-time PCR platform), a nucleic acid sequencing platform, an isothermal amplification platform (e.g., loop-mediated isothermal amplification (LAMP, RT-LAMP)), a hybridization and/or multi-signal coded (e.g., fluorescence) detector platform, etc., a nucleic acid mass spectrometry platform, a magnetic resonance platform, northern blotting, and combinations thereof (e.g., a combination of a PCR and isothermal amplification—see, e.g., Varlamov et al., “Combinations of PCR and Isothermal Amplification Techniques Are Suitable for Fast and Sensitive Detection of SARS-CoV-2 Viral RNA,” Front. Bioeng. Biotechnol., 2020).

In some embodiments, the platform is configured to measure gene expression levels semi-quantitatively, that is, rather than measuring in discrete or absolute expression, the expression levels are measured as an estimate and/or relative to each other or a specified marker or markers (e.g., expression of another, “standard” or “reference,” gene).

In some embodiments, semi-quantitative measuring includes “real-time PCR” by performing PCR cycles until a signal indicating the specified mRNA is detected, and using the number of PCR cycles needed until detection to provide the estimated or relative expression levels of the genes within the signature.

A real-time PCR platform includes, for example, a TaqMan® Low Density Array (TLDA), in which samples undergo multiplexed reverse transcription, followed by real-time PCR on an array card with a collection of wells in which real-time PCR is performed. See Kodani et al. 2011, J. Clin. Microbial. 49(6):2175-2182. A real-time PCR platform also includes, for example, a Biocartis Idylla™ sample-to-result technology, in which cells are lysed, DNA/RNA extracted, real-time PCR is performed and results are detected.

A magnetic resonance platform includes, for example, T2 Biosystems® T2 Magnetic Resonance (T2MR®) technology, in which molecular targets may be identified in biological samples without the need for purification.

The terms “array,” “microarray” and “micro array” are interchangeable and refer to an arrangement of a collection of nucleotide sequences presented on a substrate. Any type of array can be utilized in the methods provided herein. For example, arrays can be on a solid substrate (a solid phase array), such as a glass slide, or on a semi-solid substrate, such as nitrocellulose membrane. Arrays can also be presented on beads, i.e., a bead array. These beads are typically microscopic and may be made of, e.g., polystyrene. The array can also be presented on nanoparticles, which may be made of, e.g., particularly gold, but also silver, palladium, or platinum. See, e.g., Nanosphere Verigene® System, which uses gold nanoparticle probe technology. Magnetic nanoparticles may also be used. Other examples include nuclear magnetic resonance microcoils. The nucleotide sequences can be DNA, RNA, or any permutations thereof (e.g., nucleotide analogues, such as locked nucleic acids (LNAs), and the like). In some embodiments, the nucleotide sequences span exon/intron boundaries to detect gene expression of spliced or mature RNA species rather than genomic DNA. The nucleotide sequences can also be partial sequences from a gene, primers, whole gene sequences, non-coding sequences, coding sequences, published sequences, known sequences, or novel sequences. The arrays may additionally comprise other compounds, such as antibodies, peptides, proteins, tissues, cells, chemicals, carbohydrates, and the like that specifically bind proteins or metabolites.

Host-derived biomarker approaches as taught herein offer the potential to fill critical diagnostic niches, including rapid (even point-of-care) detection of one or multiple pathogen classes at once. In some embodiments, detection may be performed by the platform in less than 48, 36, or 24 hours. In some embodiments, detection may be performed by the platform in less than 22, 20, or 16 hours. In some embodiments, detection may be performed by the platform in less than 12, 10, or 8 hours. In some embodiments, detection may be performed by the platform in less than 6, 4, or 2 hours. In some embodiments, detection may be performed by the platform in less than 60, 45, or 30 minutes. Particular examples of such platforms may include, but are not limited to, PCR-based platforms.

In some embodiments, the classifier generating comprises iteratively: (i) assigning a weight for each normalized gene expression value, entering the weight and expression value for each gene into a classifier (e.g., a linear regression classifier) equation and determining a score for outcome for each of the plurality of subjects, then (ii) determining the accuracy of classification for each outcome across the plurality of subjects, and then (iii) adjusting the weight until accuracy of classification is optimized, to provide said bacterial classifier, viral classifier, fungal classifier, and/or control classifier for the platform, wherein genes having a non-zero weight are included in the respective classifier, and optionally uploading components of each classifier (genes, weights and/or etiology threshold value) onto one or more databases.

In another embodiment, the classifier comprises a linear regression classifier and the generating comprises converting a score of the classifier to a probability.

In another embodiment, the method further comprises validating the classifier against a known dataset comprising at least two relevant clinical attributes.

Another aspect of the present disclosure provides a fungal, viral, bacterial and/or control classifier made according to the methods of the present disclosure in which the classifier(s) comprise expression levels of 5, 10, 15, 20, 25, or 30 to 50, 60, 70, 80, 90 or all 94 of the genes (measurable, e.g., with oligonucleotide probes homologous to said genes) listed in Tables 1 to 5. (Note that one gene—TMEM199—appears in both the fungal and viral classifiers of Tables 1 and 3, respectively, though with a negative coefficient (weight) in the fungal classifier and a positive coefficient (weight) in the viral classifier.) Genome reference: Homo sapiens GRCh38, release 96, downloaded 2019-06-15 from: ftp.ensembl.org/pub/release-96/fasta/homo_sapiens/dna/. Transcript reference: Homo sapiens GRCh38, release 96, downloaded from here: ftp.ensembl.org/pub/release-96/gtf/homo_sapiens/. For example, the classifier(s) may comprise expression levels of from 1, 5, 10, 15, or 20 to 30, 40, 50, 60 or 70 genes of those listed in Tables 1 to 5.

TABLE 1
Fungal Classifier

| Gene | Coefficient | Ensembl ID | Full Gene Name |
|---|---|---|---|
| PPP2R2D | −1.2590 | ENSG00000175470 | Protein Phosphatase 2 Regulatory Subunit |
| | | | B, Delta |
| SNX11 | −0.8176 | ENSG00000002919 | Sorting Nexin 11 |
| ZSCAN18 | −0.3273 | ENSG00000121413 | Zinc Finger And SCAN Domain |
| | | | Containing 18 |
| ZNF701 | −0.1877 | ENSG00000167562 | Zinc Finger Protein 701 |
| KCTD6 | −0.1842 | ENSG00000168301 | Potassium Channel Tetramerization |
| | | | Domain Containing 6 |
| MTMR11 | −0.1469 | ENSG00000014914 | Myotubularin Related Protein 11 |
| SLC25A25 | −0.1176 | ENSG00000148339 | Solute Carrier Family 25 Member 25 |
| KCNC4 | −0.0767 | ENSG00000116396 | Potassium Voltage-Gated Channel |
| | | | Subfamily C Member 4 |
| LINC01232 | −0.0751 | ENSG00000280734 | Long Intergenic Non-Protein Coding |
| | | | RNA 1232 |
| NEO1 | −0.0730 | ENSG00000067141 | Neogenin 1 |
| CCNJL | −0.0421 | ENSG00000135083 | Cyclin J Like |
| HCG27 | −0.0387 | ENSG00000206344 | HLA Complex Group 27 |
| METTL2A | −0.0254 | ENSG00000087995 | Methyltransferase 2A, Methylcytidine |
| CDKNIC | −0.0166 | ENSG00000129757 | Cyclin Dependent Kinase Inhibitor 1C |
| ALGIL13P | −0.0152 | ENSG00000253981 | ALGI Like 13, Pseudogene |
| TMEM199 | −0.0098 | ENSG00000244045 | Transmembrane Protein 199 |
| TMEM158 | 0.0050 | ENSG00000249992 | Transmembrane Protein 158 |
| ARHGEF12 | 0.0158 | ENSG00000196914 | Rho Guanine Nucleotide Exchange Factor |
| | | | 12 |
| RNASE3 | 0.0197 | ENSG00000169397 | Ribonuclease A Family Member 3 |
| JHDM1D-AS1 | 0.0377 | ENSG00000260231 | KDM7A Divergent Transcript |
| (KDM7A-DT) | | | |
| SCD | 0.0565 | ENSG00000099194 | Stearoyl-CoA Desaturase |
| LY6G5C | 0.0582 | ENSG00000204428 | Lymphocyte Antigen 6 Family Member |
| | | | G5C |
| IGKV2-24 | 0.1147 | ENSG00000241294 | Immunoglobulin Kappa Variable 2-24 |
| NEDD4L | 0.1155 | ENSG00000049759 | NEDD4 Like E3 Ubiquitin Protein Ligase |
| EZH2 | 0.1774 | ENSG00000106462 | Enhancer Of Zeste 2 Polycomb |
| | | | Repressive Complex 2 Subunit |
| AZU1 | 0.2982 | ENSG00000172232 | Azurocidin 1 |
| MKI67 | 0.4134 | ENSG00000148773 | Marker Of Proliferation Ki-67 |
| RN7SL1 | 0.4808 | ENSG00000276168 | RNA Component Of Signal Recognition |
| | | | Particle 7SL1 |
| ITGA2B | 0.5095 | ENSG00000005961 | Integrin Subunit Alpha 2b |

TABLE 2
Bacterial Classifier

| Gene | Coefficient | Ensembl ID | Full Gene Name |
|---|---|---|---|
| DCAF15 | −2.0930 | ENSG00000132017 | DDB1 And CUL4 Associated Factor 15 |
| PTP4A3 | −0.4332 | ENSG00000184489 | Protein Tyrosine Phosphatase 4A3 |
| PHF1 | −0.4090 | ENSG00000112511 | PHD Finger Protein 1 |
| SSBP2 | −0.1625 | ENSG00000145687 | Single Stranded DNA Binding Protein 2 |
| DCP1B | −0.1122 | ENSG00000151065 | Decapping MRNA 1B |
| BHLHE40 | −0.1071 | ENSG00000134107 | Basic Helix-Loop-Helix Family Member |
| | | | E40 |
| AC110285.2 | −0.0988 | ENSG00000262877 | — |
| FAM234A | −0.0031 | ENSG00000167930 | Family With Sequence Similarity 234 |
| | | | Member A |
| PORCN | −0.0030 | ENSG00000102312 | Porcupine O-Acyltransferase |
| HDAC4 | 0.0017 | ENSG00000068024 | Histone Deacetylase 4 |
| SAP30L | 0.0311 | ENSG00000164576 | SAP30 Like |
| C3AR1 | 0.0715 | ENSG00000171860 | Complement C3a Receptor 1 |
| ITGA7 | 0.1458 | ENSG00000135424 | Integrin Subunit Alpha 7 |
| FAM160A2 | 0.3264 | ENSG00000051009 | FHF Complex Subunit HOOK Interacting |
| | | | Protein 1B |
| LINC01002 | 0.3378 | ENSG00000282508 | Long Intergenic Non-Protein Coding RNA |
| | | | 1002 |
| CD59 | 0.3617 | ENSG00000085063 | CD59 Molecule (CD59 Blood Group) |
| SDHC | 0.7463 | ENSG00000143252 | Succinate Dehydrogenase Complex Subunit |
| | | | C |
| DNASE1 | 1.2465 | ENSG00000213918 | Deoxyribonuclease 1 |

TABLE 3
Viral Classifier

| Gene | Coefficient | Ensembl ID | Full Gene Name |
|---|---|---|---|
| MT-RNR2 | −0.5201 | ENSG00000210082 | Mitochondrially Encoded 16S RRNA |
| VPS29 | −0.3985 | ENSG00000111237 | VPS29 Retromer Complex Component |
| MMD | −0.1855 | ENSG00000108960 | Monocyte To Macrophage Differentiation |
| | | | Associated |
| IZUMO4 | −0.1820 | ENSG00000099840 | IZUMO Family Member 4 |
| AC015912.3 | −0.1795 | ENSG00000274213 | — |
| ATP5MD | −0.0969 | ENSG00000173915 | ATP Synthase Membrane Subunit K |
| TMEM170B | −0.0669 | ENSG00000205269 | Transmembrane Protein 170B |
| SNHG8 | −0.0008 | ENSG00000269893 | Small Nucleolar RNA Host Gene 8 |
| CCDC71 | 0.0270 | ENSG00000177352 | Coiled-Coil Domain Containing 71 |
| BTBD9 | 0.0543 | ENSG00000183826 | BTB Domain Containing 9 |
| PBDC1 | 0.0712 | ENSG00000102390 | Polysaccharide Biosynthesis Domain |
| | | | Containing 1 |
| CMPK2 | 0.1287 | ENSG00000134326 | Cytidine/Uridine Monophosphate Kinase 2 |
| TMEM199 | 0.1691 | ENSG00000244045 | Transmembrane Protein 199 |
| ISG15 | 0.2129 | ENSG00000187608 | ISG15 Ubiquitin Like Modifier |
| HERC6 | 0.2211 | ENSG00000138642 | HECT And RLD Domain Containing E3 |
| | | | Ubiquitin Protein Ligase Family Member 6 |
| DDA1 | 0.2320 | ENSG00000130311 | DET1 And DDB1 Associated 1 |
| LY6E | 0.5983 | ENSG00000160932 | Lymphocyte Antigen 6 Family Member E |
| MAGED2 | 0.6030 | ENSG00000102316 | MAGE Family Member D2 |
| PIGT | 0.8054 | ENSG00000124155 | Phosphatidylinositol Glycan Anchor |
| | | | Biosynthesis Class T |

TABLE 4
SIRS Classifier

| Gene | Coefficient | Ensembl ID | Full Gene Name |
|---|---|---|---|
| BCL7B | −1.1828 | ENSG00000106635 | BAF Chromatin Remodeling Complex |
| | | | Subunit BCL7B |
| DENND4B | −1.0940 | ENSG00000198837 | DENN Domain Containing 4B |
| GABBR1 | −0.8862 | ENSG00000204681 | Gamma-Aminobutyric Acid Type B |
| | | | Receptor Subunit 1 |
| CASZ1 | −0.6972 | ENSG00000130940 | Castor Zinc Finger 1 |
| LIMK1 | −0.5658 | ENSG00000106683 | LIM Domain Kinase 1 |
| EML2 | −0.2528 | ENSG00000125746 | EMAP Like 2 |
| RCN1 | −0.1811 | ENSG00000049449 | Reticulocalbin 1 |
| EPS8L1 | −0.0867 | ENSG00000131037 | EPS8 Like 1 |
| AC136475.9 | −0.0624 | ENSG00000270972 | — |
| AIM2 | −0.0609 | ENSG00000163568 | Absent In Melanoma 2 |
| RPS28P7 | −0.0366 | ENSG00000227097 | Ribosomal Protein S28 Pseudogene 7 |
| NUMBL | −0.0024 | ENSG00000105245 | NUMB Like Endocytic Adaptor Protein |
| CCR4 | 0.0049 | ENSG00000183813 | C-C Motif Chemokine Receptor 4 |
| AC020916.1 | 0.0890 | ENSG00000267519 | miR-23a/27a/24-2 cluster host gene |
| (MIR23 AHG) | | | |
| NRG1 | 0.1894 | ENSG00000157168 | Neuregulin 1 |
| RELL1 | 0.3038 | ENSG00000181826 | RELT Like 1 |
| WIPI2 | 0.4801 | ENSG00000157954 | WD Repeat Domain, Phosphoinositide |
| | | | Interacting 2 |
| MAP1LC3B2 | 0.5365 | ENSG00000258102 | Microtubule Associated Protein 1 Light |
| | | | Chain 3 Beta 2 |
| SLC35E1 | 1.0725 | ENSG00000127526 | Solute Carrier Family 35 Member El |

TABLE 5
Healthy Classifier

| Gene | Coefficient | Ensembl ID | Full Gene Name |
|---|---|---|---|
| NPLOC4 | −2.3323 | ENSG00000182446 | NPL4 Homolog, Ubiquitin |
| | | | Recognition Factor |
| PSMD7 | −0.7541 | ENSG00000103035 | Proteasome 26S Subunit, Non-ATPase |
| | | | 7 |
| CTSB | −0.4249 | ENSG00000164733 | Cathepsin B |
| AC007342.3 | 0.0771 | ENSG00000260078 | MPHOSPH10 Pseudogene 1 |
| (MPHOSPH10P1) | | | |
| CLEC2B | 0.1127 | ENSG00000110852 | C-Type Lectin Domain Family 2 |
| | | | Member B |
| CDK5RAP3 | 0.1645 | ENSG00000108465 | CDK5 Regulatory Subunit Associated |
| | | | Protein 3 |
| RPS24 | 0.3447 | ENSG00000138326 | Ribosomal Protein S24 |
| TAF1C | 0.4309 | ENSG00000103168 | TATA-Box Binding Protein |
| | | | Associated Factor, RNA Polymerase I |
| | | | Subunit C |
| MAP3K7CL | 0.6798 | ENSG00000156265 | MAP3K7 C-Terminal Like |
| SNRNP70 | 0.6839 | ENSG00000104852 | Small Nuclear Ribonucleoprotein Ul |
| | | | Subunit 70 |

For example, a fungal classifier may comprise 3, 5, 8, 10, 12, 15, 18, 20, 25, or all 29 of the genes listed in Table 1; bacterial classifier may comprise 3, 5, 8, 10, 12, 15, or all 18 of the genes listed in Table 2; a viral classifier may comprise 3, 5, 8, 10, 12, 15, 18, or all 19 of the genes listed in Table 3; a SIRS classifier may comprise 3, 5, 8, 10, 12, 15, 18, or all 19 of the genes listed in Table 4; and/or a healthy classifier may comprise 3, 4, 5, 6, 7, 8, 9, or all 10 of the genes listed in Table 5.

One or more of these classifiers may be included in carrying out the methods taught by the present disclosure, including, but not limited to, only the fungal classifier; the fungal classifier and the bacterial classifier; the fungal classifier and the viral classifier; the fungal, bacterial and viral classifiers; the fungal and non-infectious illness (SIRS) classifiers; the fungal and healthy classifiers; the fungal, SIRS and healthy classifiers; the fungal, bacterial, viral, and SIRS classifiers; the fungal, bacterial, viral, and healthy classifiers; and the fungal, bacterial, viral, SIRS and healthy classifiers. As an example, a method may include use of a fungal classifier and a bacterial classifier in order to determine the presence of absence of a fungal and bacterial infection. As another example, a method may include use of a fungal classifier and a SIRS classifier in order to determine the presence of absence of a fungal infection and a non-infectious illness in the subject. As another example, a method may include use of a fungal classifier, a bacterial classifier and a SIRS classifier in order to determine the presence of absence of a fungal infection, bacterial infection and a non-infectious illness in the subject.

Another aspect of the present disclosure provides a fungal, viral, bacterial and/or control classifier made according to the methods of the present disclosure in which the classifier(s) comprise expression levels of 5, 10, 15, 20, 25, 30, or all 33 of the genes (measurable, e.g., with oligonucleotide probes homologous to said genes) listed in Tables 6 to 10. Genes overlapping with the classifier examples of Tables 1 to 5 are highlighted in bold type.

TABLE 6
Fungal Classifier

| Gene | Coefficient | Ensembl ID | Full Gene Name |
|---|---|---|---|
| CYTH1 | −0.2615 | ENSG00000108669 | Cytohesin 1 |
| CXCR2 | −0.0715 | ENSG00000180871 | C-X-C motif chemokine receptor 2 |
| ITGA2B | 0.1104 | ENSG00000005961 | Integrin Subunit Alpha 2b |
| MKI67 | 0.1587 | ENSG00000148773 | Marker Of Proliferation Ki-67 |
| AZU1 | 0.1907 | ENSG00000172232 | Azurocidin 1 |

TABLE 7
Bacterial Classifier

| Gene | Coefficient | Ensembl ID | Full Gene Name |
|---|---|---|---|
| HDAC4 | 0.2327 | ENSG00000068024 | Histone Deacetylase 4 |
| JAK3 | 0.0579 | ENSG00000105639 | Janus kinase 3 |
| DCAF15 | −0.6655 | ENSG00000132017 | DDB1 And CUL4 Associated Factor 15 |
| SDHC | 0.8588 | ENSG00000143252 | Succinate Dehydrogenase Complex |
| | | | Subunit C |
| GALNT2 | 0.0566 | ENSG00000143641 | Polypeptide N- |
| | | | acetylgalactosaminyltransferase 2 |
| SAP30L | 0.1857 | ENSG00000164576 | SAP30 Like |
| MCEMP1 | 0.0744 | ENSG00000183019 | Mast Cell Expressed Membrane Protein |
| | | | 1 |
| PTPN1 | 0.2036 | ENSG00000196396 | Protein Tyrosine Phosphatase Non- |
| | | | Receptor Type 1 |
| DNASE1 | 0.0181 | ENSG00000213918 | Deoxyribonuclease 1 |

TABLE 8
Viral Classifier

| Gene | Coefficient | Ensembl ID | Full Gene Name |
|---|---|---|---|
| PIGT | 0.4754 | ENSG00000124155 | Phosphatidylinositol Glycan Anchor |
| | | | Biosynthesis Class T |
| TPT1 | −0.1809 | ENSG00000133112 | Tumor Protein, Translationally- |
| | | | controlled 1 |
| HERC6 | 0.2741 | ENSG00000138642 | HECT And RLD Domain Containing |
| | | | E3 Ubiquitin Protein Ligase Family |
| | | | Member 6 |
| MRPL49 | 0.0372 | ENSG00000149792 | Mitochondrial Ribosomal Protein L49 |
| LY96 | −0.0129 | ENSG00000154589 | Lymphocyte Antigen 96 |
| LY6E | 0.2987 | ENSG00000160932 | Lymphocyte Antigen 6 Family Member |
| | | | E |
| CCDC71 | 0.0859 | ENSG00000177352 | Coiled-Coil Domain Containing 71 |
| SPATS2L | 0.0196 | ENSG00000196141 | Spermatogenesis Associated Serine |
| | | | Rich 2 Like |

TABLE 9
SIRS Classifier

| Gene | Coefficient | Ensembl ID | Full Gene Name |
|---|---|---|---|
| SLC35E1 | 0.3314 | ENSG00000127526 | Solute Carrier Family 35 Member |
| | | | E1 |
| CASZ1 | −0.3204 | ENSG00000130940 | Castor Zinc Finger 1 |
| WIPI2 | 0.2381568 | ENSG00000157954 | WD Repeat Domain, |
| | | | Phosphoinositide Interacting 2 |
| FAM131A | 0.0001 | ENSG00000175182 | Family With Sequence Similarity |
| | | | 131 Member A |
| RELL1 | 0.2343 | ENSG00000181826 | RELT Like 1 |
| GABBR1 | −0.315788 | ENSG00000204681 | Gamma-Aminobutyric Acid Type |
| | | | B Receptor Subunit 1 |
| MAP1LC3B2 | 0.0138 | ENSG00000258102 | Microtubule Associated Protein 1 |
| | | | Light Chain 3 Beta 2 |

TABLE 10
Healthy Classifier

| Gene | Coefficient | Ensembl ID | Full Gene Name |
|---|---|---|---|
| E2F2 | −0.0540 | ENSG00000007968 | E2F Transcription Factor 2 |
| RPS24 | 0.2333 | ENSG00000138326 | Ribosomal Protein S24 |
| CTSB | −0.3401 | ENSG00000164733 | Cathepsin B |
| CLK2 | 0.5041 | ENSG00000176444 | CDC Like Kinase 2 |

Another aspect of the present disclosure provides a fungal, viral, bacterial and/or control classifier made according to the methods of the present disclosure in which the classifier(s) comprise expression levels of the genes in bold type listed in Tables 6 to 10. That is, a fungal classifier comprises ITGA2B, MK167, and AZU1 (each with a positive coefficient); a bacterial classifier comprises HDAC4, DCAF15, SDHC, SAP30L, and DNASE1 (each with a positive coefficient), and DCAF15 (with negative coefficient); a viral classifier comprises PIGT, HERC6 and LY6E (each with a positive coefficient); a SIRS classifier comprises SLC35E1, WIPI2, RELL1, and MAP1LC3B2 (each with a positive coefficient), and CASZ1 and GABBR1 (each with a negative coefficient; and a healthy classifier comprises RPS24 (with a positive coefficient) and CTSB (with a negative coefficient).

As noted above, one or more of these classifiers may be included in carrying out the methods taught by the present disclosure, including, but not limited to, only the fungal classifier; the fungal classifier and the bacterial classifier; the fungal classifier and the viral classifier; the fungal, bacterial and viral classifiers; the fungal and non-infectious illness (SIRS) classifiers; the fungal and healthy classifiers; the fungal, SIRS and healthy classifiers; the fungal, bacterial, viral, and SIRS classifiers; the fungal, bacterial, viral, and healthy classifiers; and the fungal, bacterial, viral, SIRS and healthy classifiers. As an example, a method may include use of a fungal classifier and a bacterial classifier in order to determine the presence of absence of a fungal and bacterial infection. As another example, a method may include use of a fungal classifier and a SIRS classifier in order to determine the presence of absence of a fungal infection and a non-infectious illness in the subject. As another example, a method may include use of a fungal classifier, a bacterial classifier and a SIRS classifier in order to determine the presence of absence of a fungal infection, bacterial infection and a non-infectious illness in the subject.

In some embodiments, the use of these signature(s) can identify multiple different illness etiologies (fungal infection such as candidemia, bacterial infection, viral infection, non-infectious illness (“SIRS”), and/or healthy) at once with a high degree of accuracy. For example, in some embodiments the etiology has an area under the receiver operating characteristic (auROC or ROC), which is the probability that a subject will have an accurately assigned etiology, of at least 0.90, such as at least 0.91, at least 0.92, at least 0.93, at least 0.94, at least 0.95, at least 0.96, at least 0.97, at least 0.98, or at least 0.99; or at least 0.80, such as at least 0.81, at least 0.82, at least 0.83, at least 0.84, at least 0.85, at least 0.86, at least 0.87, at least 0.88, or at least 0.89. As known in the art, an auROC of 0.80 means that the correct assignment will be made 80% of the time, and an auROC above 0.80 is considered to be an excellent performance of the classifier.

As aspect of the present invention is a method for classifying a subject, comprising: (a) obtaining a biological sample from the subject; (b) measuring on a platform a signature indicative of a fungal infection, and optionally one or more of a bacterial infection, a viral infection, healthy and/or non-infectious illness in the biological sample, said signature(s) comprising gene expression levels of a pre-defined set of genes; (c) entering the gene expression levels into a fungal classifier, and optionally one or more additional classifiers selected from a bacterial infection classifier, a viral classifier, and a control classifier (healthy and/or non-infectious illness), said classifier(s) comprising pre-defined weighting values (i.e., coefficients) for each of the genes of the pre-defined set of genes for the platform; and (d) classifying the subject as having a fungal infection, and/or a bacterial infection, a viral infection, or a control, based upon said gene expression levels and the classifier(s). In some embodiments, the method comprises normalizing the gene expression levels to generate normalized gene expression values, and the entering comprises entering the normalized gene expression values into the classifier(s); and the classifying comprises calculating the probability for the fungal infection, and optionally a bacterial infection, a viral infection, or a control based upon said normalized gene expression values and the classifier(s). In some embodiments, the method further comprises generating a report assigning the subject a score indicating the probability of the fungal infection, and optionally the bacterial infection, viral infection, healthy and/or non-infectious illness. In some embodiments, the method further comprises: (e) administering an appropriate therapy to the subject based on classifying.

Another aspect of the present disclosure provides a method for diagnosing and/or treating a fungal infection such as candidemia in a subject suffering therefrom, or at risk thereof, comprising, consisting of, or consisting essentially of: (a) obtaining a biological sample from the subject; (b) measuring on a platform gene expression levels of a pre-defined set of genes (i.e., signature) in the biological sample; (c) optionally normalizing the gene expression levels to generate normalized gene expression values; (d) entering the normalized gene expression values into one or more classifiers selected from a bacterial infection classifier, a viral classifier, a fungal classifier, and/or a control classifier, said classifier(s) comprising pre-defined weighting values (i.e., coefficients) for each of the genes of the pre-defined set of genes for the platform, optionally wherein said classifier(s) are retrieved from one or more databases; (e) calculating the probability for one or more of a bacterial, viral, and, fungal, and/or control based upon said normalized gene expression values and the classifier(s), to thereby determine whether presence of a fungal infection such as candidemia in the subject, or the likelihood of the subject developing such a fungal infection; and (f) optionally, administering an appropriate therapy.

In some embodiments, the method further comprises generating a report assigning the subject a score indicating the probability of the fungal infection such as candidemia.

In some embodiments, the pre-defined set of genes comprises expression levels of 5, 10, 15, 20, 25, or 30 to 50, 60, 70, 80, 90 or all 94 of the genes listed in Tables 1 to 5. For example, the classifier(s) may comprise expression levels of from 1, 5, 10, 15, or 20 to 30, 40, 50, 60 or 70 genes of those listed in Tables 1 to 5.

As examples, the pre-defined set may comprise 3, 5, 8, 10, 12, 15, 18, 20, 25, or all 29 of the genes listed in Table 1; and optionally 3, 5, 8, 10, 12, 15, or all 18 of the genes listed in Table 2; 3, 5, 8, 10, 12, 15, 18, or all 19 of the genes listed in Table 3; 3, 5, 8, 10, 12, 15, 18, or all 19 of the genes listed in Table 4; and/or 3, 4, 5, 6, 7, 8, 9, or all 10 of the genes listed in Table 5, in any combination.

As another example, the pre-defined list of genes may comprise expression levels of 5, 10, 15, 20, 25, 30, or all 33 of the genes listed in Tables 6 to 10. As a further example, the pre-defined list of genes may comprise expression levels of the genes in bold type listed in Tables 6 to 10.

In some embodiments, the biological sample is selected from the group consisting of peripheral blood, sputum, nasopharyngeal swab, nasopharyngeal wash, bronchoalveolar lavage, endotracheal aspirate, cerebrospinal fluid, urine, and combinations thereof. In certain embodiments, the biological sample comprises a peripheral blood sample. In certain embodiments, the biological sample comprises a bronchoalveolar lavage.

### Classification Systems

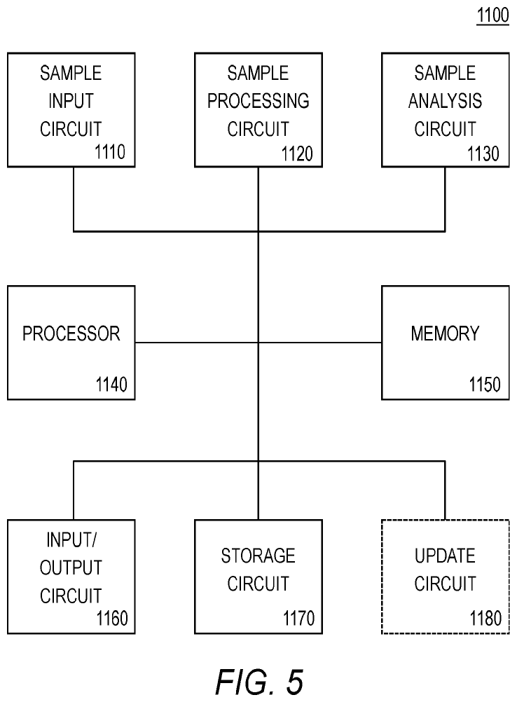

With reference to FIG. 5, a classification system and/or computer program product 1100 may be used in or by a platform, according to various embodiments described herein. A classification system and/or computer program product 1100 may be embodied as one or more enterprise, application, personal, pervasive and/or embedded computer systems that are operable to receive, transmit, process and store data using any suitable combination of software, firmware and/or hardware and that may be standalone and/or interconnected by any conventional, public and/or private, real and/or virtual, wired and/or wireless network including all or a portion of the global communication network known as the Internet, and may include various types of tangible, non-transitory computer readable medium.

As shown in FIG. 5, the classification system 1100 may include a processor subsystem 1140, including one or more Central Processing Units (CPU) on which one or more operating systems and/or one or more applications run. While one processor 1140 is shown, it will be understood that multiple processors 1140 may be present, which may be either electrically interconnected or separate. Processor(s) 1140 are configured to execute computer program code from memory devices, such as memory 1150, to perform at least some of the operations and methods described herein, and may be any conventional or special purpose processor, including, but not limited to, digital signal processor (DSP), field programmable gate array (FPGA), application specific integrated circuit (ASIC), and multi-core processors.

The memory subsystem 1150 may include a hierarchy of memory devices such as Random Access Memory (RAM), Read-Only Memory (ROM), Erasable Programmable Read-Only Memory (EPROM) or flash memory, and/or any other solid state memory devices. A storage circuit 1170 may also be provided, which may include, for example, a portable computer diskette, a hard disk, a portable Compact Disk Read-Only Memory (CDROM), an optical storage device, a magnetic storage device and/or any other kind of disk- or tape-based storage subsystem. The storage circuit 1170 may provide non-volatile storage of data/parameters/classifiers for the classification system 1100. The storage circuit 1170 may include disk drive and/or network store components. The storage circuit 1170 may be used to store code to be executed and/or data to be accessed by the processor 1140. In some embodiments, the storage circuit 1170 may store databases which provide access to the data/parameters/classifiers used for the classification system 1110 such as the signatures, weights, thresholds, etc. Any combination of one or more computer readable media may be utilized by the storage circuit 1170. The computer readable media may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. As used herein, a computer readable storage medium may be any tangible medium that can contain, or store a program for use by or in connection with an instruction execution system, apparatus, or device.

An input/output circuit 1160 may include displays and/or user input devices, such as keyboards, touch screens and/or pointing devices. Devices attached to the input/output circuit 1160 may be used to provide information to the processor 1140 by a user of the classification system 1100. Devices attached to the input/output circuit 1160 may include networking or communication controllers, input devices (keyboard, a mouse, touch screen, etc.) and output devices (printer or display). The input/output circuit 1160 may also provide an interface to devices, such as a display and/or printer, to which results of the operations of the classification system 1100 can be communicated so as to be provided to the user of the classification system 1100.

An optional update circuit 1180 may be included as an interface for providing updates to the classification system 1100. Updates may include updates to the code executed by the processor 1140 that are stored in the memory 1150 and/or the storage circuit 1170. Updates provided via the update circuit 1180 may also include updates to portions of the storage circuit 1170 related to a database and/or other data storage format which maintains information for the classification system 1100, such as the signatures, weights, thresholds, etc.

The sample input circuit 1110 of the classification system 1100 may provide an interface for the platform as described hereinabove to receive biological samples to be analyzed. The sample input circuit 1110 may include mechanical elements, as well as electrical elements, which receive a biological sample provided by a user to the classification system 1100 and transport the biological sample within the classification system 1100 and/or platform to be processed. The sample input circuit 1110 may include a bar code reader that identifies a bar-coded container for identification of the sample and/or test order form. The sample processing circuit 1120 may further process the biological sample within the classification system 1100 and/or platform so as to prepare the biological sample for automated analysis. The sample analysis circuit 1130 may automatically analyze the processed biological sample. The sample analysis circuit 1130 may be used in measuring, e.g., gene expression levels of a pre-defined set of genes with the biological sample provided to the classification system 1100. The sample analysis circuit 1130 may also generate normalized gene expression values by normalizing the gene expression levels. The sample analysis circuit 1130 may retrieve from the storage circuit 1170 a fungal infection classifier, and optionally also one or more of a viral infection classifier, a bacterial infection classifier, a non-infectious illness classifier, and a healthy subjects classifier. The sample analysis circuit 1130 may enter the normalized gene expression values into the classifier(s). The sample analysis circuit 1130 may calculate an etiology probability or likelihood for a fungal infection, and optionally also one or more of a viral infection, a bacterial infection, a non-infectious illness, and a healthy subject based upon said classifier(s) and control output, via the input/output circuit 1160.

The sample input circuit 1110, the sample processing circuit 1120, the sample analysis circuit 1130, the input/output circuit 1160, the storage circuit 1170, and/or the update circuit 1180 may execute at least partially under the control of the one or more processors 1140 of the classification system 1100. As used herein, executing “under the control” of the processor 1140 means that the operations performed by the sample input circuit 1110, the sample processing circuit 1120, the sample analysis circuit 1130, the input/output circuit 1160, the storage circuit 1170, and/or the update circuit 1180 may be at least partially executed and/or directed by the processor 1140, but does not preclude at least a portion of the operations of those components being separately electrically or mechanically automated. The processor 1140 may control the operations of the classification system 1100, as described herein, via the execution of computer program code.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages.

The program code may execute entirely on the classification system 1100, partly on the classification system 1100, as a stand-alone software package, partly on the classification system 1100 and partly on a remote computer or entirely on the remote computer or server. In the latter scenario, the remote computer may be connected to the classification system 1100 through any type of network, including a local area network (LAN) or a wide area network (WAN), or the connection may be made to an external computer (for example, through the Internet using an Internet Service Provider) or in a cloud computer environment or offered as a service such as a Software as a Service (SaaS).

In some embodiments, the system includes computer readable code that can transform quantitative, or semi-quantitative, detection of gene expression to a cumulative score or probability of the etiology of a fungal infection, and optionally also one or more of a viral infection, a bacterial infection, a non-infectious illness, and a healthy subject.

In some embodiments, the system is a sample-to-result system, with the components integrated such that a user can simply insert a biological sample to be tested, and some time later (preferably a short amount of time, e.g., up to 30 or 45 minutes, or up to 1, 2, or 3 hours, or up to 8, 12, 24 or 48 hours) receive a result output from the system.

Another aspect of the present disclosure provides all that is described and illustrated herein.

The following Examples are provided by way of illustration and not by way of limitation.

### EXAMPLES

### Example 1. The Host Transcriptional Response to Candidemia is Dominated by Neutrophil Activation and Heme Biosynthesis and Supports Novel Diagnostic Approaches

### A. Methods

Subject Enrollment: All study patients were enrolled after informed consent at Duke University Medical Center (DUMC). The study was approved by the Institutional Review Board (IRB) at DUMC (Pro00083484) and was performed in accordance with the Declaration of Helsinki. Forty-eight hospitalized patients with candidemia were enrolled through the Infectious Diseases Data and Specimen Repository program at Duke University (Durham, NC) at the time of first blood culture positivity for Candida spp. Whole blood was collected from these subjects in PAXGene tubes for RNA sequencing and serum was collected from each subject for additional analysis. Each subject with candidemia had at least 1 and at most 14 samples collected over the course of the study. RNA sequencing data from previously enrolled subjects presenting to the Emergency Department with viral, bacterial, or non-infectious illness (from DUMC, Durham VA Health Care System, UNC Health Care, and Henry Ford Hospital) were also run with the candidemia samples. Peripheral blood samples were also similarly collected from a population of non-hospitalized healthy controls. Clinical adjudication served as the reference standard, which was performed after enrollment but prior to gene expression measurements. The adjudication process used here has been previously described. Non-infectious subjects were labeled as a systemic inflammatory response syndrome (SIRS) phenotype—defined by at least two SIRS criteria (temperature <360 Celsius (C) or >38° C., tachycardia >90 beats per minute, tachypnea >20 breaths per minute or PaCO2<32 mmHg, white cell count <4,000 cells/mm3 or >12,000 cells/mm3 or >10% neutrophil band forms) without evidence of infection.

RNA extraction, library preparation, and sequencing: Total RNA was extracted from human blood preserved and stored in PAXgene Blood RNA Tubes using the Qiagen PAXgene Blood miRNA Kit according to the manufacturer's protocol. RNA quantity and quality were assessed using the Nanodrop 2000 spectrophotometer (Thermo Scientific) and Agilent 2100 Bioanalyzer, respectively. RNA sequencing libraries were generated using NuGEN Universal mRNA-seq kit with AnyDeplete Globin (NuGEN Technologies, Redwood City, CA) and sequenced on the Illumina NovaSeq 6000 instrument with S2 flow cell and 50 bp paired-end reads (performed through the Duke Sequencing and Genomic Technologies Core)

RNA sequencing data processing: For both the discovery and validation datasets, RNA sequences were mapped to the human genome (hg) and gene expression quantified using STAR with parameters: quantMode: ‘GeneCounts’; outSAMtype: ‘None’; outSAMmode: ‘None’; readFilesCommand: ‘zcat’ and ENSEMBL gene reference Homo sapiens GRCh38 DNA, release 96, downloaded from: ftp://ftp.ensembl.org/pub/release-96/fasta/homo_sapiens/dna/ (for gene quantification). All other parameters were left at their default values for STAR version 2.7.1a. Samples with a low number of mapped reads (<12 million reads) or low average pairwise correlation (<0.70) were excluded from analyses. In the discovery cohort, genes with 0 counts or counts/million <2 in≥50% of samples were excluded. The validation cohort was reduced to the set of genes passing quality control in the discovery cohort. The remaining gene counts were normalized using TMM, within each cohort.

### Statistical Analysis

Differential expression: For both the discovery and validation datasets, the R Bioconductor package limma was used to estimate the mean expression for each outcome group: Candidemia, Bacterial, Viral, SIRS, and healthy, while adjusting for age, sex, and race, using the empirical Bayesian linear modeling with voom weights. Generalized linear hypothesis testing (i.e., contrasts) was used to test for differential expression between specific infection-type groups (i.e., candidemia vs. healthy). A false discovery rate of less than 5% was used to determine statistical significance for each comparison. The differential expression results from the discovery and validation cohorts were combined using inverse-variance weighted meta-analysis of the log 2 fold changes with a cohort random effect, as implemented in the R package meta.

Diagnostic classifier development and validation: Regularized multinomial logistic regression (lasso), implemented in the R package glmnet was used to identify a multi-gene signature of infection type. Three different unbiased feature selections were used prior to constructing the model: 1) top 1000 most variable genes, 2) top 2000 most variable genes, 3) all 11,100 genes that passed quality control. The multinomial model performance was estimated using nested leave one sample out cross validation (LOOCV) as follows: for each sample, one sample was held out and the remaining samples were used to estimate the model. Within the (N-1) samples, 10-fold cross validation was used to optimize the sparsity parameter. The optimal sparsity parameter was then used to estimate the model in the N-1 samples. The resulting model was used to estimate the predicted class probabilities in the held-out samples. After completing the LOOCV, the predicted class probabilities from the held-out samples were used to assess the training performance metrics: per-class auROC, confusion matrices, overall sensitivity, and overall specificity. The overall model was estimated using all data with the sparsity parameter optimized through 10-fold cross validation of the discovery dataset. This overall model was used to predict infection class probabilities in other sequenced samples from other datasets. Model testing performance metrics included per-class area under the Receiver Operating Characteristics curves (auROCs) and confusion matrices.

Additional Validation: Independent, external validation was performed with two human microarray gene expression datasets. For the Ramilo dataset, Affymetrix CEL files and sample characteristics were downloaded from GEO (GSE6269-GPL96). CEL files were imported and processed using the R Bioconductor packages readAffy. Expression values were normalized using germa. Probes detected in fewer than four samples and Affymetrix control probes were excluded. For the Tsalik dataset, Affymetrix microarray gene expression was previously processed and normalized, as previously described. For both the Ramilo and Tsalik datasets, microarray probes were mapped to ensemble gene identifiers and reduced to the subset of probes that mapped to the classifier gene list. Resulting expression values were log 2 transformed and analyzed using the same regularized multinomial modeling, cross validation procedure, and performance metrics used in the discovery analysis to re-estimate the model weights.

Additional validation was performed with an in vitro PBMC microarray dataset consisting of viral (influenza), bacterial (Escherichia coli and Streptococcus pneumoniae) and fungal (Candida albicans, Cryptococcus neoformans and gattii) infections of healthy human PBMCs. Similar to the Ramilo and Tsalik datasets, .CEL files were imported and processed using the R Bioconductor package readAffy, normalized using germa, and lowly expressed probes, defined as detected in less than four samples, and control probes were excluded. Microarray probe identifiers were mapped to ensemble genes; data was reduced to the subset of probes that mapped to the classifier gene list; and log 2 transformed. The same regularized multinomial modeling, cross validation procedure, and performance metrics used in the discovery analysis were applied here to estimate the classifier model on a different gene expression platform.

Biological Pathway Analysis: Gene lists were analyzed using the Database for Annotation, Visualization and Integrated Discovery (DAVID, www.david.abcc.ncifcrf.gov) to identify significantly enriched pathways. We also applied weighted gene co-expression network analysis (WGCNA) to the discovery dataset (i.e., 11,131 genes in 136 samples). Using these parameters: power parameter=6; UPGMA clustering; dynamic tree cutting with method=“hyprid”, deepSplit=2, and minclustersize=30, we identified 41 clusters (or “modules”). The aggregate expression of all genes assigned to a module can be summarized using PCA, where the 1st principal component (named eigengene) is used as a summary measure of module gene expression. Because each module eigengene can be thought of as the aggregate expression of all of the genes in that module, we can use the eigengene value to test for association with infection type. Each module eigengene was tested for association with Candidemia infection using linear regression. Modules with parameter estimates with a Benjamini-Hochburg adjusted p-value <5% were considered statistically significant. Additionally, each module was assessed for enrichment of KEGG and GO pathways using functions goana and kegga available in the R bioconductor package limma. Ensembl gene identifiers were mapped to entrez gene identifiers and enrichment was assessed for the set of genes within the module compared to all genes that passed quality control and mapped to an entrez gene. Enrichment p-values were adjusted for multiple testing within each module using the Benjamini-Hochberg adjustment.

Beta-D-glucan testing: Serum samples from all subjects with candidemia, 5 healthy subjects, and 20 subjects with viral infection underwent BDG testing (Viracor Eurofins) (range <31 to >500). Values of >500 were processed as 501 and values <31 were processed as previously described. AuROCs were calculated for the BDG test values and the candidemia component of the gene expression signature, separately for the discovery and validation cohorts, restricted to the subset of subjects with both BDG testing and gene expression. BDG and gene expression auROCs were compared using the DeLong test. BDG and gene expression data were also compared by Spearman correlation. Mann-Whitney test was used for comparison of means.

### B. Results

i. Study Population

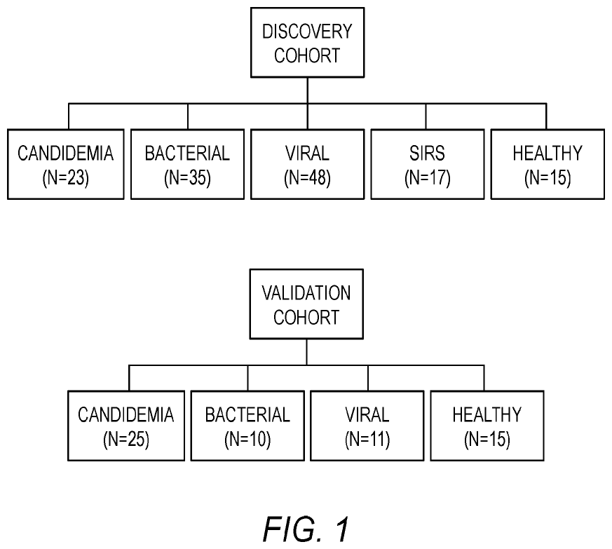

Forty-eight hospitalized adult subjects were enrolled at the time of first blood culture positivity for Candida spp. from 2011 to 2014 at Duke University Medical Center (a minimum of 2 days after initial blood culture collection), along with serial sampling on a subset of patients. In addition, we enrolled patients with similar clinical backgrounds but with proven acute respiratory viral infection, acute bacterial (pneumonia or bacteremia) infection, or clinically adjudicated non-infectious illness, as well as uninfected healthy subjects (n=151, FIG. 1). The study included subjects from a variety of clinical backgrounds, including solid organ transplants, stem cell transplants, hematologic malignancies, patients in the LCU with central venous catheters, and others. A total of 7 different Candida spp. were identified, most commonly C. albicans and C. glabrata.

TABLE 11
Clinical Information on Candidemic Subjects

| | Candidemia | Candidemia | |
|---|---|---|---|
| Clinical Manifestations, | Discovery Cohort | Validation Cohort | |
| Labs, and Treatment | (n = 23) | (n = 25) | p value |
| Additional Sites of Infection | | | |
| Eyes | 1 | 1 | |
| Heart | 1 | 0 | |
| Hepatosplenic | 0 | 0 | |
| Peritonitis | 0 | 1 | |
| Esophagus | 0 | 2 | |
| CNS | 0 | 0 | |
| Lungs (empyema) | 3 | 4 | |
| Genitourinary | 0 | 2 | |
| Soft tissue | 0 | 0 | |
| Bone | 0 | 0 | |
| None | 13 | 15 | |
| Unknown | 5 | 0 | |
| Candida spp.* | | | |
| C. albicans | 9 | 4 | |
| C. glabrata | 7 | 7 | |
| C. parapsilosis | 5 | 3 | |
| C. tropicalis | 2 | 9 | |
| C. krusei | 1 | 2 | |
| C. dubliniensis | 0 | 1 | |
| C. zeylanoides | 0 | 1 | |
| Initial Antifungal | | | |
| Fluconzole | 9 | 2 | |
| Micafungin | 12 | 22 | |
| Voriconazole | 0 | 0 | |
| Isavuconazole | 0 | 0 | |
| Posaconazole | 0 | 0 | |
| Amphotericin | 2 | 1 | |
| Final Antifungal | | | |
| Fluconzole | 8 | 10 | |
| Micafungin | 7 | 13 | |
| Voriconazole | 0 | 0 | |
| Isavuconazole | 0 | 0 | |
| Posaconazole | 0 | 0 | |
| Amphotericin | 1 | 1 | |
| Combination therapy | 3 | 1 | |
| Unknown | 4 | 0 | |
| Number of hospitalized | 11.94 ± 13.94 days | 12.60 ± 17.55 days | p = 0.73 |
| days pre-dx (mean ± SD) | (range 0-50) | (range 0-75) | |
| Total duration of | 41.39 ± 51.50 days | 28.32 ± 28.13 days | p = 0.32 |
| hospitalization (mean ± SD) | (range 5-221) | (range 4-109) | |
| Fever at time of Dx** | 10 | 15 | |
| Hypothermia at time of Dx | 1 | 0 | |

*Two subjects had simultaneous infection with more than one Candida species.
**Nine subjects had limited medical records, and temperature was not recorded.

ii. Discovery and Validation Cohorts

Subjects and controls were divided at random into discovery and validation cohorts for initial analysis. The discovery cohort and validation cohorts included 138 subjects and 61 subjects, respectively (FIG. 1). In the discovery cohort, 23 subjects were adjudicated as having bloodstream infection with Candida spp. in the absence of other types of infection. Thirty-five subjects were included with confirmed bacterial infection and 48 with confirmed viral infection (both monomicrobial) as controls. Additionally, as patients may also present clinically with acute non-infectious diseases, 17 subjects were included with acute non-infectious illness, labeled as systemic inflammatory response syndrome (SIRS). In the validation cohort there were 25 subjects with candidemia, along with 10 subjects with confirmed bacterial infection and 11 subjects with confirmed viral infection (both monomicrobial). Fifteen healthy subjects were also included in each cohort as controls—the mean age of the healthy controls was 20.9 years in the discovery dataset and 33.5 years in the validation dataset. Sixty-five percent of the candidemic subjects in the discovery cohort and 80% in the validation cohort were on antifungal treatment at the time of initial sampling.

iii. The Transcriptional Response to Candidemia is Robust and Reveals Antifungal Defense Mechanisms.

Candidemia triggered a strong transcriptomic response in human hosts with 1,641 genes differentially up-regulated compared to healthy controls. These up-regulated genes corresponded to known components of the host immune response to fungal infection, including innate immune responses, defense response to fungus, leukocyte migration, and response to yeast. Other stress-associated pathways included response to cytokine, inflammatory response, cellular response to oxidative stress, and host regulation of heme synthesis and iron metabolism. There were 2,316 down-regulated genes clustered into immune processes such as adaptive immune response, regulation of immune response, B cell proliferation, humoral immune response, immunoglobulin production, and T cell co-stimulation. To further elucidate how transcriptomic responses define active biological pathways in the host, weighted gene co-expression network analysis (WGCNA) was performed to identify clusters of correlated genes associated with candidemia compared to healthy controls. Clusters significantly upregulated in candidemia included pathways of immune activation and inflammation, including innate immune response and neutrophil activation, migration, and degranulation.

iv. The Transcriptional Response to Candidemia is Unique Compared to Other Infectious Triggers.

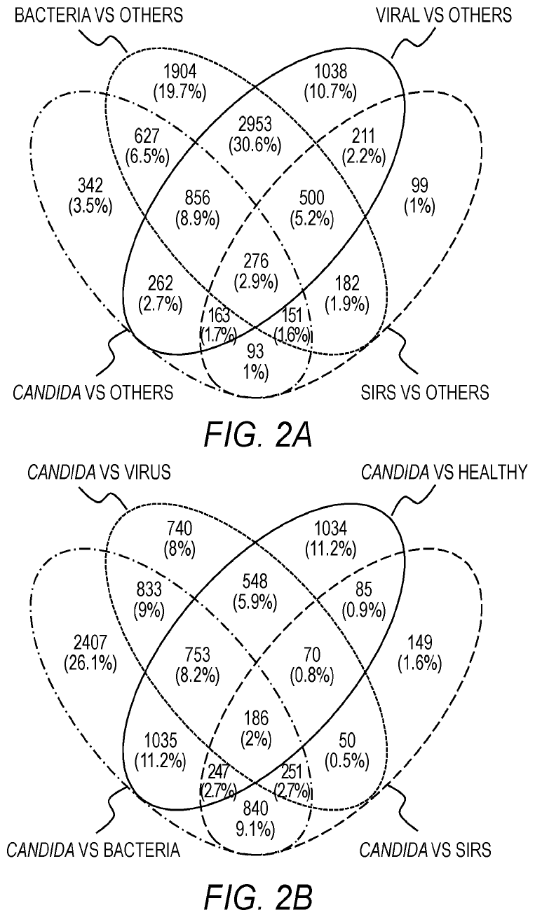

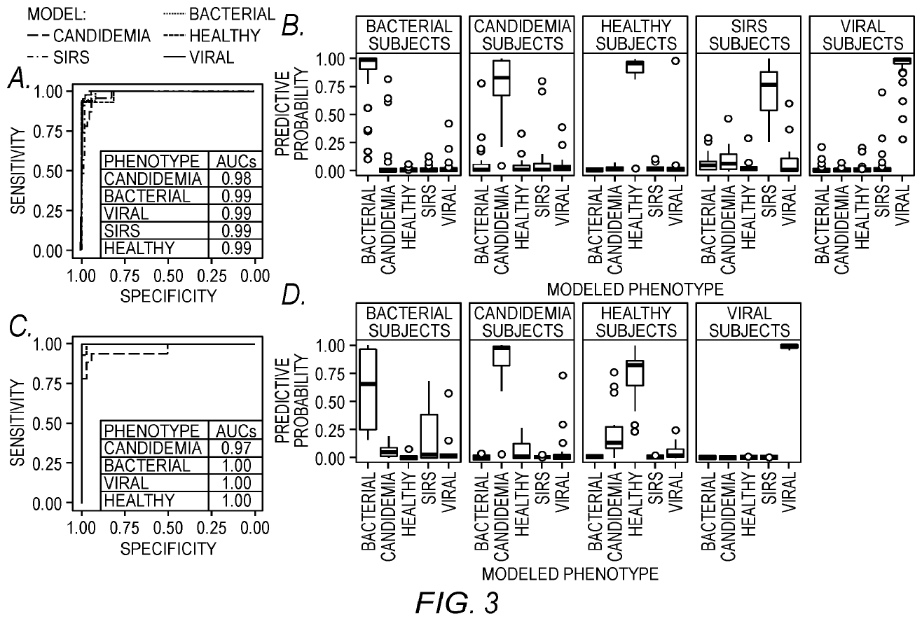

In addition to healthy controls, univariate comparisons were also performed between the transcriptomic responses to candidemia and acute bacterial and viral infection as well as non-infectious SIRS. While there were some conserved components of the host response observed across infection phenotypes, there were also 342 (12%) genes uniquely differentially expressed during candidemia compared to all others. When examining the differential expression of genes for Candida compared to other clinical phenotypes, the largest distinction was seen between candidemia and bacterial infection (2,407 unique genes) followed by viral infection and SIRS (740 and 149 genes, respectively) (FIG. 2A-2B). This highlights that the transcriptional response to candidemia has unique features compared to other classes of infection. Interestingly, when the transcriptomic response to candidemia was compared to that of other pathogen classes, the top genes up-regulated in candidemia again clustered into pathways weighted toward neutrophil activation and heme biosynthesis, further highlighting the strength of these responses during fungal infection.

v. A Multinomial Gene Expression Classifier Distinguishes Candidemia from Viral or Bacterial Infection.

Regularized multinomial logistic regression analyses was next used to determine a set of genes (“signature”) that was most consistently co-regulated across samples from each group of infected subjects. For Candida infection, prior work in a mouse model demonstrated that gene expression signatures discriminate early and late invasive candidiasis and that signal intensity decreases over time. Thus, for development of a diagnostic classifier, we utilized only the first RNA sample obtained for each Candida subject after initial blood culture positivity (median 5 days, range 2-23 days). All other acute infection phenotypes only had one RNA sample per subject per episode, taken at the time of initial presentation with their respective infections.

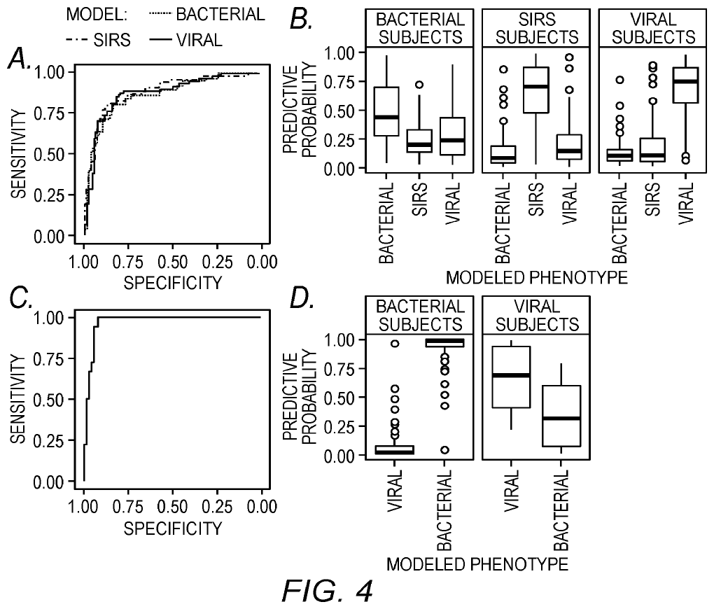

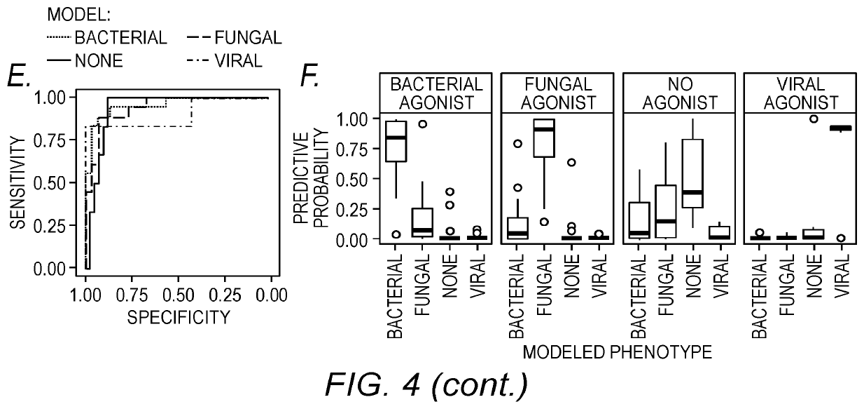

Model performance was assessed with auROCs and confusion matrices for all infection classes. All performance measures were cross-validated. A 94-gene classifier was identified that could accurately distinguish candidemia, bacterial, viral, SIRS, and healthy phenotypes. (FIG. 3) AuROCs were 0.98 (95% CI 0.96-1) for candidemia, 0.99 (95% CI 0.98-1) for both the bacterial and viral infection, 0.99 (95% CI 0.97-1) for SIRS, and 0.99 (95% CI 0.96-1) for healthy subjects (FIG. 4, Supplemental Table 7). The signature derived from the discovery cohort was then used to predict infection class in the validation dataset. Per-class auROCs and confusion matrices were computed. Performance in the validation cohort was equally good: auROCs were 0.97 (95% CI 0.90-1) for candidemia, and 1 for bacterial infection (95% CI 1-1), viral infection (95% CI 1-1), and healthy subjects (95% CI 0.99-1).

vi. A Blood-Based Gene Expression Signature of Candidemia is Maximally Expressed at Peak Illness and Decreases in Intensity Over Time.

Once a Candida-specific diagnostic signature was identified, it was sought to examine signal intensity over time as discrimination between early and late disease and defining response to treatment can have an impact on a patient's clinical care, treatment options, and prognosis. A total of 28 subjects with candidemia had samples collected at more than one date after culture positivity, ranging from 2 to 14 samples per subject. Samples were collected 2 to 80 days from initial culture. When comparing quantitative levels of expression of genes in the signature for these subjects we found that the overall trend in signal intensity decreased from first to last time-point in subjects with isolated candidemia. However, there was marked variability in quantitative signal strength and time to resolution between subjects. There was an expected inverse correlation seen between quantitative gene expression and days from positive blood culture (p=−0.441, p=0.0009). In several subjects where appropriate samples were available, the signature-derived predicted probability of candidemia decreased over time with therapy, and eventually those subjects were predicted by the model to be healthy once candidemia had resolved.

Given the uniqueness of this dataset and lack of public gene expression data on candidemic subjects, for validation we next applied the classifier to two independent gene expression data sets from human subjects with acute bacterial and viral illnesses (Ramilo, et al. and Tsalik, et al.) (FIG. 4). When applied to the Ramilo, et al. dataset, the novel classifier performed well with an auROC 0.97 (0.95% CI 0.94-1). When applied to the Tsalik, et al. dataset, auROCs were 0.87 (95% CI 0.80-0.93) for bacterial infection, 0.88 for viral (95% CI 0.82-0.92), and 0.89 (95% CI 0.84-0.94) for non-infectious illness.

Next, the candidemia results were compared to gene expression data from an in vitro stimulation assay whereby peripheral blood mononuclear cells (PBMCs) were isolated from healthy individuals and then exposed to pathogens from multiple classes. In this model, cells were then harvested at 24 hours post-exposure to analyze transcriptomic responses during experimental viral (influenza), bacterial (Streptococcus pneumonia or Escherichia coli), and fungal (Candida albicans or Cryptococcus neoformans or gattii) infections. The human candidemia classifier was then applied to these data, where it accurately identified the relevant pathogen exposure—auROCs were 0.94 (95% CI 0.88-0.99) for fungal infection, 0.96 (95% CI 0.89-1) for bacterial, 0.90 (95% CI 0.69-1) for viral infection, and 0.94 (95% CI 0.86-0.99) for healthy control cells (FIG. 4).

vii. Comparison to BDG

It was next sought to compare the diagnostic accuracy of serum BDG levels with the novel transcriptomic biomarker signature. The mean level of BDG at the time of first blood culture positivity for candidemia was 246 pg/mL±192 (range <31 to >500), which was not significantly higher than the mean for last BDG at 235 pg/mL±189 (range <31 to >500, p=0.85) Serial BDG measurements showed that only 43% (13/30) of subjects had decreasing values of BDG in response to treatment, and the rate of decrease was highly variable. The overall BDG auROC was 0.90 (95% CI 0.80-0.97). When broken down into discovery and validation cohorts, the candidemia component of the gene expression classifier had higher performance characteristics than BDG, though this result was not statistically significant. The discovery auROC for gene expression was 1 (95% CI 1-1) compared to 0.98 (95% CI 0.94-1) for BDG (p=0.39), the validation auROC was 0.94 (95% CI 0.81-1) for gene expression compared to 0.83 (95% CI 0.63-0.97) for BDG (p=0.35). BDG level was found to be moderately inversely correlated with days from positive blood culture (ρ=−0.29, p=0.05) and mildly correlated with quantitative gene expression (ρ=0.258, p=0.084).

### C. Discussion

Multiple pathogen-based diagnostic modalities for candidemia are currently available but often hindered by delayed time-to-result and/or suboptimal sensitivity and specificity. Host-derived biomarker approaches offer the potential to fill critical diagnostic niches, including rapid (even point-of-care) detection of multiple pathogen classes at once, and improved specificity through identification of pathologic host responses. In this work, we have for the first time defined the host response to candidemia as seen through the lens of the transcriptome in circulating leukocytes. This has enabled the development of a host signature able to differentiate acute fungal infection from viral, bacterial, and SIRS phenotypes that may also cause similar acute illness in at-risk hosts.

The host response to Candida infection has both shared and unique features compared to other pathogen classes, and this is manifested at the transcriptional level in peripheral blood. Over 1,600 differentially expressed genes (DEGs) were found in the presence of candidemia compared to healthy controls. Many of these DEGs reflected known components of the immune response to fungal infection or critical illness while such cytokine signaling, inflammatory responses, and cellular responses to oxidative stress. Some, like neutrophil activation and migration, are known to play a role in antifungal defense, but the strength of these responses, even when compared to similarly ill subjects with acute bacterial infections, was surprising and highlights the critical importance of these pathways in clearing Candida spp. Other enriched pathways identify potentially novel host response mechanisms to Candida infection such as alterations in the regulation of heme synthesis. While iron is known to be critical for fungal pathogens such as Candida in vitro, the results suggest the human host may manipulate this system as part of the response to fungal infection.

Through multinomial logistic regression analyses we identified a unifying signature that could model the host response to multiple different illness etiologies at once with a high degree of accuracy (auROC 0.98 for candidemia). The candidemia component of this classifier performed better than the standard of care diagnostic BDG test. Importantly, the candidemia signature exhibited strong performance despite over 70% of the cohort being on active empiric antifungal treatment at the time of initial testing, a common clinical approach that impairs many traditional pathogen detection strategies such as blood culture. Furthermore, the classifier performs well across a wide array of typical clinical backgrounds including neutropenia and multiple types of immunosuppression, as well as across 7 different Candida species. Another advantage to the multinomial approach presented here is that a single test can inform diagnosis of multiple conditions (i.e., fungal, bacterial, viral, SIRS, healthy) simultaneously.

One limitation of this study is that while the in silico and in vitro validation data support generalizability, this was a single-center study and will require validation in other candidemic populations once additional cohorts/datasets are available. While the cohort is diverse, the relatively small candidemia sample size limits sub-group analysis, and further work with larger groups of neutropenic and other types of immunocompromised patients will be necessary. Additionally, the study design limits our ability to identify test performance at earlier times during Candida infection where treatment may be most efficacious, as subjects were not enrolled until after their blood cultures had turned positive. While this study defines the performance of the transcriptomic signature for the diagnosis of candidemia, it is not known how such a signature performs in or is impacted by the presence of other fungal diseases such as invasive mold infections. Finally, this study did not directly evaluate the performance of the signature in cases of invasive candidiasis (esophageal, abdominal, etc.) without candidemia, so the signal strength and efficacy in these infections will need to be formally explored.

### D. Conclusion

The host response to candidemia in hospitalized adults is highly conserved and is distinct from the transcriptomic responses to acute viral and bacterial infection. Clinic-ready platforms capable of operationalizing PCR-based signatures of the sizes demonstrated herein already exist, offering a proximal pathway to clinical application of these findings. Harnessing these pathogen class-specific responses allows for better understanding of the immunopathogenesis of fungal infections in human hosts and shows promise for the development of host gene expression-based assays to simultaneously differentiate multiple types of clinical illnesses in acutely ill patients.

### Example 2. Performance of Fungal Classifier in Cryptococcus Infections

As noted above in Example 1, we compared the candidemia results to gene expression data from an in vitro stimulation assay whereby peripheral blood mononuclear cells (PBMCs) were isolated from healthy individuals and then exposed to pathogens from multiple classes. In this model, cells were then harvested at 24 h post-exposure to analyze transcriptomic responses during experimental viral (influenza), bacterial (Streptococcus pneumonia or Escherichia coli), and fungal (Candida albicans or Cryptococcus neoformans or gattii) infections. We then applied the human candidemia classifier to these data, and it accurately identified the relevant pathogen exposure-auROCs were 0.94 (95% CI 0.88-0.99) for fungal infection, 0.96 (95% CI 0.89-1) for bacterial, 0.90 (95% CI 0.69-1) for viral infection, and 0.94 (95% CI 0.86-0.99) for healthy control cells (FIG. 4).

To further clarify the distinction in signature performance between Candida and Cryptococcus, we examined the predictive probabilities and confusion matrix at the agonist level. We observed that there was not a statistically significant difference between Candida and Cryptococcus (ANOVA F test p value=0.2866).

Therefore, the fungal classifier trained with Candida infection samples was able to identify other fungal infections such as those from Cryptococcus, supporting its use to identify fungal infections more generally.

### Example 3. Additional Example Classifiers

A reduced-sized gene expression signature was generated using the same lasso logistic regression with nested cross validation procedure used to generate the full model as described in Example 1 above, with one modification: the lasso model was specified such that the maximum number of features, or genes, in the model is 40. The resulting classifiers are presented in Table 12.

TABLE 12
Reduced Size Classifiers

| Ensembl ID | Gene | Bacterial | Fungal | Healthy | SIRS | Viral |
|---|---|---|---|---|---|---|
| ENSG00000108669 | CYTH1 | 0 | −0.2615 | 0 | 0 | 0 |
| ENSG00000180871 | CXCR2 | 0 | −0.0715 | 0 | 0 | 0 |
| ENSG00000007968 | E2F2 | 0 | 0 | −0.0540 | 0 | 0 |
| ENSG00000068024 | HDAC4 | 0.2327 | 0 | 0 | 0 | 0 |
| ENSG00000105639 | JAK3 | 0.0579 | 0 | 0 | 0 | 0 |
| ENSG00000124155 | PIGT | 0 | 0 | 0 | 0 | 0.4754 |
| ENSG00000127526 | SLC35E1 | 0 | 0 | 0 | 0.3314 | 0 |
| ENSG00000130940 | CASZ1 | 0 | 0 | 0 | −0.3204 | 0 |
| ENSG00000132017 | DCAF15 | −0.6655 | 0 | 0 | 0 | 0 |
| ENSG00000133112 | TPT1 | 0 | 0 | 0 | 0 | −0.1809 |
| ENSG00000138326 | RPS24 | 0 | 0 | 0.2333 | 0 | 0 |
| ENSG00000138642 | HERC6 | 0 | 0 | 0 | 0 | 0.2741 |
| ENSG00000143252 | SDHC | 0.8588 | 0 | 0 | 0 | 0 |
| ENSG00000143641 | GALNT2 | 0.0566 | 0 | 0 | 0 | 0 |
| ENSG00000149792 | MRPL49 | 0 | 0 | 0 | 0 | 0.0372 |
| ENSG00000154589 | LY96 | 0 | 0 | 0 | 0 | −0.0129 |
| ENSG00000157954 | WIPI2 | 0 | 0 | 0 | 0.2382 | 0 |
| ENSG00000160932 | LY6E | 0 | 0 | 0 | 0 | 0.2987 |
| ENSG00000164576 | SAP30L | 0.1857 | 0 | 0 | 0 | 0 |
| ENSG00000164733 | CTSB | 0 | 0 | −0.3401 | 0 | 0 |
| ENSG00000175182 | FAM131A | 0 | 0 | 0 | 0.0001 | 0 |
| ENSG00000176444 | CLK2 | 0 | 0 | 0.5041 | 0 | 0 |
| ENSG00000177352 | CCDC71 | 0 | 0 | 0 | 0 | 0.0859 |
| ENSG00000181826 | RELL1 | 0 | 0 | 0 | 0.2343 | 0 |
| ENSG00000183019 | MCEMP1 | 0.0744 | 0 | 0 | 0 | 0 |
| ENSG00000196141 | SPATS2L | 0 | 0 | 0 | 0 | 0.0196 |
| ENSG00000196396 | PTPN1 | 0.2036 | 0 | 0 | 0 | 0 |
| ENSG00000204681 | GABBR1 | 0 | 0 | 0 | −0.3158 | 0 |
| ENSG00000213918 | DNASE1 | 0.0181 | 0 | 0 | 0 | 0 |
| ENSG00000258102 | MAP1LC3B2 | 0 | 0 | 0 | 0.0138 | 0 |
| ENSG00000005961 | ITGA2B | 0 | 0.1104 | 0 | 0 | 0 |
| ENSG00000148773 | MKI67 | 0 | 0.1587 | 0 | 0 | 0 |
| ENSG00000172232 | AZU1 | 0 | 0.1907 | 0 | 0 | 0 |

As noted above, the reduced-size gene signature was newly-created using the same process as the reported in Example 1, but with a limit on the gene numbers involved. This can lead to some variation in genes between signatures. As such, it is not just a subset of the original signature, though some genes do appear in both.

One skilled in the art will readily appreciate that the present disclosure is well adapted to carry out the objects and obtain the ends and advantages mentioned, as well as those inherent therein. The present disclosure described herein is representative of preferred embodiments, which are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The foregoing is illustrative of the present invention, and is not to be construed as limiting thereof. The invention is defined by the following claims, with equivalents of the claims to be included therein.

1. A method for classifying a subject, comprising:
(a) obtaining a biological sample from the subject;
(b) measuring on a platform a signature indicative of a fungal infection, and optionally one or more of a bacterial infection, a viral infection, healthy and/or non-infectious illness in the biological sample, said signature(s) comprising gene expression levels of a pre-defined set of genes;
(c) entering the gene expression levels into a fungal classifier, and optionally one or more additional classifiers selected from a bacterial infection classifier, a viral classifier, and a control classifier (healthy and/or non-infectious illness), said classifier(s) comprising pre-defined weighting values (i.e., coefficients) for each of the genes of the pre-defined set of genes for the platform; and
(d) classifying the subject as having a fungal infection, and/or a bacterial infection, a viral infection, or a control, based upon said gene expression levels and the classifier(s).

2. The method of claim 1, wherein the method comprises normalizing the gene expression levels to generate normalized gene expression values, and the entering comprises entering the normalized gene expression values into the classifier(s); and
the classifying comprises calculating the probability for the fungal infection, and optionally a bacterial infection, a viral infection, or a control based upon said normalized gene expression values and the classifier(s).

3. The method according to claim 2 in which the method further comprises generating a report assigning the subject a score indicating the probability of the fungal infection, and optionally the bacterial infection, viral infection, healthy and/or non-infectious illness.

4. The method according to claim 1, further comprising: (e) administering an appropriate therapy to the subject based on the classifying.

5. The method according to claim 1 in which the pre-defined set of genes is a set of from 1, 5, 10, 15, or 20 to 30, 40, 50, 60 or 70 genes.

6. The method according to claim 1 in which the pre-defined set of genes is a set of from 1, 5, 10, 15, or 20 to 30, 40, 50, 60 or 70 genes listed in Tables 1-5.

7. The method according to claim 1 in which the pre-defined set of genes is a set of from 1, 5, or 10, to 15, 20, 25, 30 or 33 genes listed in Tables 6-10 (e.g., selected from the genes listed in bold type in Tables 6-10).

8. The method according to claim 1 in which the subject has symptoms of an infection (e.g., fever).

9. The method according to claim 1 in which the subject has symptoms of sepsis.

10. The method according to claim 1 in which the biological sample is selected from the group consisting of peripheral blood, sputum, cerebrospinal fluid, urine, nasopharyngeal swab, nasopharyngeal wash, bronchoalveolar lavage, endotracheal aspirate, and combinations thereof.

11. The method according to claim 1 in which the biological sample comprises a peripheral blood sample.

12. The method according to claim 1 in which the biological sample comprises a bronchoalveolar lavage.

13. The method according to claim 1 in which the measuring comprises or is preceded by one or more steps of: purifying cells from the sample, breaking the cells of the sample, and isolating RNA from the sample.

14. The method according to claim 1 in which the measuring comprises PCR amplification, isothermal amplification, sequencing and/or nucleic acid probe hybridization.

15. The method according to claim 1 in which the platform comprises an array platform, a thermal cycler platform (e.g., multiplexed and/or real-time PCR platform), a hybridization and multi-signal coded (e.g., fluorescence) detector platform, a nucleic acid mass spectrometry platform, a nucleic acid sequencing platform, an isothermal amplification platform, or a combination thereof.

16. The method according to claim 1, wherein the fungal infection comprises a yeast, such as Candida, Trichosporon, or Cryptococcus.

17. The method according to claim 1 in which the fungal classifier was produced by a process comprising: (i) obtaining a biological sample from a plurality of subjects known to be suffering from a fungal infection; (ii) obtaining a biological sample from a plurality of non-hospitalized healthy controls and/or a plurality of subjects known to be suffering from a non-infectious illness; (iii) measuring on the platform the gene expression levels of a plurality of genes in each of the samples from steps (i) and (ii); (iv) normalizing the gene expression levels obtained in step (iii) to generate normalized gene expression values; and (f) generating the fungal classifier.

18. The method according to claim 1 in which the fungal classifier was produced by a process comprising: (i) obtaining a biological sample from a plurality of subjects known to be suffering from a fungal infection; (ii) obtaining a biological sample from a plurality of subjects known to be suffering from a bacterial infection; (iii) measuring on the platform the gene expression levels of a plurality of genes in each of the samples from steps (i) and (ii); (iv) normalizing the gene expression levels obtained in step (iii) to generate normalized gene expression values; and (f) generating the fungal classifier.

19. The method according to claim 1 in which the fungal classifier was produced by a process comprising: (i) obtaining a biological sample from a plurality of subjects known to be suffering from a fungal infection; (ii) obtaining a biological sample from a plurality of subjects known to be suffering from a viral infection; (iii) measuring on the platform the gene expression levels of a plurality of genes in each of the samples from steps (i) and (ii); (iv) normalizing the gene expression levels obtained in step (iii) to generate normalized gene expression values; and (f) generating the fungal classifier.

20. The method as in claim 17 in which the generating comprises iteratively: (i) assigning a weight for each normalized gene expression value, entering the weight and expression value for each gene into a classifier (e.g., a linear regression classifier) equation and determining a score for outcome for each of the plurality of subjects, then (ii) determining the accuracy of classification for each outcome across the plurality of subjects, and then (iii) adjusting the weight until accuracy of classification is optimized, to provide said fungal classifier, bacterial classifier, viral classifier, and/or control classifier for the platform, wherein genes having a non-zero weight are included in the respective classifier, and optionally uploading components of each classifier (genes, weights and/or etiology threshold value) onto one or more databases.

21. A method for detecting a fungal infection in a subject, comprising:
providing a biological sample of the subject; and
measuring on a platform differential expression of a pre-defined set of genes, said pre-defined set of genes comprising 5, 10, 15, 20, 25, or 30 to 50, 60, 70, 80, 90 or all 94 of the genes listed in Tables 1 to 5; such as 3, 5, 8, 10, 12, 15, 18, 20, 25, or all 29 of the genes listed in Table 1; and optionally 3, 5, 8, 10, 12, 15, or all 18 of the genes listed in Table 2; 3, 5, 8, 10, 12, 15, 18, or all 19 of the genes listed in Table 3; 3, 5, 8, 10, 12, 15, 18, or all 19 of the genes listed in Table 4; and/or 3, 4, 5, 6, 7, 8, 9, or all 10 of the genes listed in Table 5,
or wherein said pre-defined set of genes comprises 5, 10, 15, 20, 25, 30, or all 33 of the genes listed in Tables 6 to 10; such as 1, 2, 3, 4 or all 5 of the genes listed in Table 6; and optionally 1, 2, 3, 4, 5, 6, 7, 8 or all 9 of the genes listed in Table 7; 1, 2, 3, 4, 5, 6, 7 or all 8 of the genes listed in Table 8; 1, 2, 3, 4, 5, 6 or all 7 of the genes listed in Table 9; and/or 1, 2, 3 or all 4 of the genes listed in Table 10,
or wherein said pre-defined set of genes comprises ITGA2B, MKI67, and AZU1; and optionally HDAC4, DCAF15, SDHC, SAP30L, DNASE1, and DCAF15; PIGT, HERC6, and LY6E; SLC35E1, WIPI2, RELL1, MAP1LC3B, CASZ1 and GABBR1; and/or RPS24 and CTSB,
wherein the differential expression of the pre-defined set of genes indicates the presence or absence of the fungal infection in the subject.

22. The method of claim 21, wherein said measuring comprises or is preceded by one or more steps of: purifying cells from said sample, breaking the cells of said sample, and isolating RNA from said sample.

23. The method of claim 21, wherein said measuring comprises semi-quantitative PCR, isothermal amplification, and/or nucleic acid probe hybridization.

24. The method of claim 21, wherein
said platform comprises an array platform, a thermal cycler platform (e.g., multiplexed and/or real-time PCR platform), an isothermal amplification platform, a hybridization and multi-signal coded (e.g., fluorescence) detector platform, a nucleic acid mass spectrometry platform, a nucleic acid sequencing platform, or a combination thereof.

25. The method of claim 21, wherein the subject is suffering from symptoms of an infection (e.g., fever).

26. The method of claim 21, wherein the subject is suffering from symptoms of sepsis.

27. The method of claim 21, said method further comprising treating said subject for the fungal infection when the presence of the fungal infection is detected.

28. A method of treating a fungal infection in a subject comprising administering to said subject an appropriate treatment regimen when said subject is determined to have a fungal infection by a method of claim 21.

29. The method of claim 28, wherein the appropriate treatment regimen comprises administering an antifungal antibiotic.

30. The method of claim 28, where the appropriate treatment regimen comprises administering a therapeutic agent selected from the group consisting of: echinocandins (e.g., caspofungin, micafungin, anidulafungin), azole antifungals (e.g., fluconazole, voriconazole, isavuconazole, posaconazole), polyenes (e.g., amphotericin B), pyrimidine analogues (e.g., 5-fluorocytosine (5-FC, or flucytosine)), APX001 (fosmanogepix), APX879, benzothioureas, clofazimine, hydrazycines (e.g., BHBM and B0), ibomycin, monoclonal antibody 18B7, resorcylate aminopyrazoles (e.g., Compound 112), sertraline, tamoxifen, VT-1598, and the like, including combinations thereof.

31. The method of claim 28, wherein the method further comprises monitoring the subject for efficacy of the appropriate treatment regimen.

32. A system for detecting a fungal infection in a subject, comprising:
at least one processor;
a sample input circuit configured to receive a biological sample from the subject;
a sample analysis circuit coupled to the at least one processor and configured to determine gene expression levels of the biological sample of a set of pre-determined genes indicative of the fungal infection;
an input/output circuit coupled to the at least one processor;
a storage circuit coupled to the at least one processor and configured to store data, parameters, and/or gene set(s); and
a memory coupled to the processor and comprising computer readable program code embodied in the memory that when executed by the at least one processor causes the at least one processor to perform operations comprising:
controlling/performing measurement via the sample analysis circuit of gene expression levels of the pre-defined set of genes in said biological sample;
normalizing the gene expression levels to generate normalized gene expression values;
retrieving from the storage circuit pre-defined weighting values (i.e., coefficients) for each of the genes of the pre-defined set of genes;
calculating a likelihood of the fungal infection based upon weighted values of the normalized gene expression values; and
controlling output via the input/output circuit of a determination of the presence or absence of the fungal infection.

33. The system of claim 32, wherein the pre-defined set of genes comprises 5, 10, 15, 20, 25, or 30 to 50, 60, 70, 80, 90 or all 94 of the genes listed in Tables 1 to 5; such as 3, 5, 8, 10, 12, 15, 18, 20, 25, or all 29 of the genes listed in Table 1; and optionally 3, 5, 8, 10, 12, 15, or all 18 of the genes listed in Table 2; 3, 5, 8, 10, 12, 15, 18, or all 19 of the genes listed in Table 3; 3, 5, 8, 10, 12, 15, 18, or all 19 of the genes listed in Table 4; and/or 3, 4, 5, 6, 7, 8, 9, or all 10 of the genes listed in Table 5,
or wherein said pre-defined set of genes comprises 5, 10, 15, 20, 25, 30, or all 33 of the genes listed in Tables 6 to 10; such as 1, 2, 3, 4 or all 5 of the genes listed in Table 6; and optionally 1, 2, 3, 4, 5, 6, 7, 8 or all 9 of the genes listed in Table 7; 1, 2, 3, 4, 5, 6, 7 or all 8 of the genes listed in Table 8; 1, 2, 3, 4, 5, 6 or all 7 of the genes listed in Table 9; and/or 1, 2, 3 or all 4 of the genes listed in Table 10,
or wherein said pre-defined set of genes comprises ITGA2B, MKI67, and AZU1; and optionally HDAC4, DCAF15, SDHC, SAP30L, DNASE1, and DCAF15; PIGT, HERC6, and LY6E; SLC35E1, WIPI2, RELL1, MAP1LC3B, CASZ1 and GABBR1; and/or RPS24 and CTSB.

34. The system of claim 32, where said system comprises computer readable code to transform quantitative, or semi-quantitative, detection of gene expression to a cumulative score or probability of the fungal infection.

35. The system of claim 32, wherein said system comprises an array platform, a thermal cycler platform (e.g., multiplexed and/or real-time PCR platform), a hybridization and multi-signal coded (e.g., fluorescence) detector platform, a nucleic acid mass spectrometry platform, a nucleic acid sequencing platform, an isothermal amplification platform, or a combination thereof.

36.-37. (canceled)
